(19) United States
(12) Patent Application Publication
(10) Pub. No.: US 2022/0162232 A1
(43) Pub. Date: May 26, 2022

(54) CAPTURE OF MICROVESICLES FOR DIAGNOSTIC PURPOSES

(71) Applicants: UNIVERSITÉ DE BORDEAUX, BORDEAUX (FR); INSTITUT POLYTECHNIQUE DE BORDEAUX, TALENCE (FR); CENTRE NATIONAL DE LA RECHERCHE SCIENTIFIQUE, PARIS (FR); CENTRE HOSPITALIER UNIVERSITAIRE DE BORDEAUX, TALENCE CEDEX (FR)

(72) Inventors: LAURENT PLAWINSKI, BELIN BELIET (FR); MARIE-CHRISTINE DURRIEU, BORDEAUX (FR); MARION PETITET, SAINT-GERMAIN-EN-LAYE (FR); JULIE LAVIE, VILLENAVE D'ORNON (FR); MARIE CHRISTINE OERTHEL, VILLENAVE D'ORNON (FR); SYLVAIN NLATE, PESSAC (FR); CHRISTEL CHANSEAU, BORDEAUX (FR); VINCENT RIGALLEAU, PESSAC (FR); LAURE ALEXANDRE, GRADIGNAN (FR)

(21) Appl. No.: 17/593,380

(22) PCT Filed: Mar. 18, 2020
(86) PCT No.: PCT/FR2020/050593

§ 371 Date: Sep. 17, 2021

(30) Foreign Application Priority Data
Mar. 19, 2019 (FR) 1902814

(51) Int. Cl. C07F 3/00; C07F 1/00; G01N 33/68

(52) U.S. Cl. CPC C07F 3/003; G01N 2800/28; G01N 33/6896; C07F 1/005

(57) ABSTRACT

The present invention relates to functionalized supports and their use in the diagnosis of pathologies.

The present invention relates to functionalized supports and the use thereof for the diagnosis of diseases.

### TECHNOLOGICAL BACKGROUND

Cellular microvesicles are vesicles released into the extracellular matrix by budding of a cell on activation following a stress. The microvesicles released into biological fluids (blood, urine, tears, etc.) contain, and/or carry on their surface, constituents of the parent cell (lipids, proteins and RNA, or mitochondrial DNA) and can therefore be regarded as early markers of a pathological state of a tissue.

A method described in international application PCT/FR2012/050610 employs a synthetic ligand for capturing phosphatidylserine-positive microvesicles.

The present invention relates to an improvement of the means and methods of capture presented in application PCT/FR2012/050610. It also relates to the identification of biomarkers of diseases of interest by means of functionalized supports according to the embodiments presented hereunder.

### SUMMARY OF THE INVENTION

The invention relates to a functionalized solid support, useful for the capture, detection and/or characterization of cellular microvesicles. The support according to the invention is also useful in the medical field for carrying out methods of diagnosis and/or prognosis, in particular for early diagnosis of a disease.

The support according to the invention is a solid support, in particular polymeric, metallic or ceramic, functionalized by means of a compound of formula (I) or (II) as described below. According to a particular embodiment, the support is a polymeric support, in particular a support made of poly(vinyl chloride) (or PVC), poly(ethylene terephthalate) (or PET), or polystyrene (or PS), functionalized by means of a compound of formula (I) or (II) as described below.

The invention also relates to a method for the diagnosis of a disease, comprising detection of the presence or absence of specific markers identified by the inventors.

This detection may in particular be carried out after capturing microvesicles according to the embodiments presented in the present application.

### LEGEND FOR THE FIGURES

FIG. 1 shows steps in the functionalization of PET.

FIG. 2 shows steps in the functionalization of PVC.

FIG. 3 shows schematically the surface of PVC at each step in the functionalization thereof with complex 1. (A) Step 1: virgin PVC. (B) Step 2: addition of ethylenediamine (EDA). (C) Step 3: addition of glutaraldehyde (Glu.). (D) Step 4: addition of complex 1 (C1).

FIG. 4 shows schematically the surface of “DNA-BIND®” at each step of its functionalization thereof with complex 1. (A) Step 1: virgin “DNA-BIND®”. (B) Step 2: addition of complex 1 (C1).

FIG. 5 is a graphical representation of the podocalyxin/beta-actin ratio in the microvesicles derived from the urine of healthy donors and diabetic patients. Each point represents the ratio obtained for one patient. The value of the mathematical mean is indicated above each group of patients. The statistical data were analyzed with a Tukey's ANOVA test. *: p<0.05; **: p<0.01 and ***: p<0.001. Legends: n=number of patients, Healthy: Healthy donors; Normo: Normoalbuminuric Patients, Micro: Microalbuminuric Patients; Macro: Macroalbuminuric Patients.

FIG. 6 is a graphical representation of the podocalyxin/annexin-A5 ratio in the microvesicles derived from the urine of healthy donors and diabetic patients. Each point represents the ratio obtained for one patient. The value of the mathematical mean is indicated above each group of patients. The statistical data were analyzed with a Tukey's ANOVA test. *: p<0.05; **: p<0.01; ***: p<0.001 and ****: p<0.0001. Legends: n=number of patients, Healthy: Healthy donors; Normo: Normoalbuminuric Patients, Micro: Microalbuminuric Patients; Macro: Macroalbuminuric Patients.

FIG. 7 shows the detection of the CD41 platelet marker and graphical representation of the CD41/annexin-A5 ratio in the microvesicles derived from the plasma of diabetic patients. A. Electrophoresis of the proteins of microvesicles derived from the plasma of two diabetic patients; one normoalbuminuric (Normo) and one macroalbuminuric (Macro). Revelation of the Western blot using antibodies against CD41, and the beta-actin and annexin-A5 reference proteins. B. Quantification of the signal from the bands relating to CD41, and annexin-A5 using the ImageJ software and graphical representation of the CD41/annexin-A5 ratio.

FIG. 8 shows detection of the enzymatic activity of uPA in the microvesicles isolated from cultured HUVEC cells. Graphical representation of the mean value of the initial rates measured in mOD/min by spectrophotometry. Legend: Unrinsed condition control=100%; rinsed condition without complex=without material; rinsed condition with complex=with material.

FIG. 9 shows immunologic detection of podocalyxin in the microvesicles isolated from human urine and captured by the material. Graphical representation of the absorbances obtained by spectrophotometry at 450 nm in different conditions: 1—Microvesicles+TMB, 2—Primary antibody+TMB, 3—Secondary antibody+TMB, 4—Primary antibody+Secondary antibody+TMB, 5—Primary antibody+Secondary antibody+TMB.

FIG. 10 shows immunologic detection of podocalyxin as a function of the concentration of microvesicles from human urine captured by the material. Graphical representation of the absorbances obtained by spectrophotometry at 450 nm as a function of the concentration of the microvesicles expressed in ng/μL. The equation of the straight line and R² are calculated using Excel software.

FIG. 11 shows immunologic detection of CD41 in the microvesicles isolated from human plasma and captured by the material. Graphical representation of the signals detected for the presence of CD41 in the microvesicles of the normoalbuminuric diabetic patients P1 and P2. The control condition (Ctl) represents the background noise recorded in the well without microvesicles in the presence of the reagent TMB. FIG. 12 shows immunologic detection of CD41 in human plasma after capture of the microvesicles by the material. Graphical representation of the signals detected for the presence of CD41 in the microvesicles of the normoalbuminuric diabetic patients P3, P4 and P5. The signals are calculated after deducting the signal obtained for the background noise (i.e. in the presence of all the reagents but without primary antibody).

FIG. 13 shows a CryoMEB analysis of the capture of the microvesicles derived from cellular activation of the HUVEC cells, by the material PVC+C1. (A) Scale bar 10 μm. (B) Scale bar 2 μm. (C) Scale bar 500 nm.

FIG. 14 shows a CryoMEB analysis of the capture of the microvesicles derived from cellular activation of the HUVEC cells, by the material “DNA-BIND®”+C1. (A) Scale bar 500 nm. (B) Scale bar 500 nm and measurement of the diameter of a microvesicle.

FIG. 15 shows a CryoMEB analysis of the capture of the microvesicles derived from a human urine sample from a healthy subject, by the material D“NA-BIND®”+C1. (A) Scale bar 5 μm. (B) Scale bar 500 nm. (C) Scale bar 500 nm and measurement of the diameter of a microvesicle.

FIG. 16 shows detection of the marker annexin-A5 on microvesicles of the HUVEC cell model at two doses (5 μg and 10 μg), in duplicate, without capture (Control, Ctrl) and after 1 h of capture on the kit at 37° C. (Kit).

FIG. 17 shows detection of the marker annexin-A5 on microvesicles of a urine sample from two healthy subjects without capture (Control, Ctrl) and after 1 night of capture by the kit at 4° C. (Kit).

FIG. 18 shows detection of the pathological biomarker podocalyxin and of the microvesicular reference marker annexin-A5 on the microvesicles of urine samples from a healthy subject and from a diabetic patient with nephropathy at the microalbuminuric stage (Micro) without capture (Control, Ctrl) and after 1 night of capture by the kit at 4° C. (Kit).

FIG. 19 shows comparison of the podocalyxin/annexin-A5 ratio corresponding to the microvesicles of urine samples from a healthy subject and from a diabetic patient with nephropathy at the microalbuminuric stage (Micro) without capture (Control, Ctrl) and after 1 night of capture by the kit at 4° C. (Kit).

FIG. 20 shows comparison of the podocalyxin/annexin-A5 ratio corresponding to the microvesicles of the urine samples from healthy subjects and from diabetic patients with nephropathy at the Normo, Micro and Macro stage without capture (Control, Ctrl) and after 1 night of capture by the kit at 4° C. (Kit).

### DETAILED DESCRIPTION OF THE INVENTION

According to a first aspect, the invention relates to a solid support, characterized in that it comprises, grafted on its surface, a compound of the following formula (I) or (II):

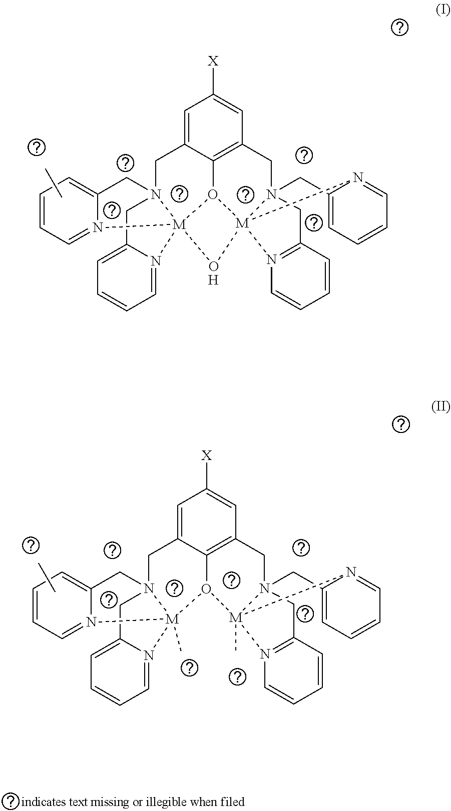

in which
M⁺ⁱ represents a metal ion and i is 1, 2 or 3;
L represents an exchangeable ligand;
X represents a group —(CH₂)ₘ—NH₂, or a group —CH₂—NHC(O)—R—NH₂ in which R is a C₂-C₁₀, in particular C₅-C₁₀, alkyl group, substituted or unsubstituted, linear or branched;
m=1 to 12;
n=1, 2 or 3; and
Y represents H or (CH₂)ₚ—NH₂, where p=0 to 12.

According to the present invention, the compound of formula (I) or (II) is grafted covalently on the solid support. The method described in application PCT/FR2012/050610 does not teach covalent grafting of the complexes to the surface of the supports. Rather, it describes noncovalent adsorption of polyglutaraldehyde on a support and then immobilization of the complex on the polyglutaraldehyde via the NH₂ functions of said complex. Advantageously, thanks to the method of the present invention, the complex is more accessible for phosphatidylserine. Moreover, the method according to the present invention gives better reproducibility of the functionalization. The method according to the present invention thus allows precise control of the density of complexes immobilized covalently, and in particular has the following advantages:
better control of the presentation of the complex for better interaction with the phosphatidylserine present in the microvesicles;
better control of the density of grafting of the complex;
no desorption is possible in the medium;
better reproducibility of the functionalization from one material to another and from batch to batch.

The compounds of formula (I) or (II) are cationic, and their counterion may be selected, for example, from the anions tosylate, nitrate, sulfate, sulfonate, thiosulfate, halide, hexafluorophosphate, tetraphenylborate, tetrafluoroborate, perchlorate, etc., in particular the perchlorate, nitrate, sulfate, halide and carbonate anions.

According to a particular embodiment, M is selected from Zn, Cu, Mn, Co, Ni and Fe, Zn or Cu being preferred, more particularly Zn.

According to a particular embodiment, Y represents H.

According to other particular embodiments:
X represents —(CH₂)ₘ—NH₂, m=1, n=1 and Y represents H; or
X represents —(CH₂)ₘ—NH₂, m=1, n=2 and Y represents H; or
X represents —CH₂—NHC(O)—R—NH₂, R represents C5H10, n=1 or 2 and Y represents H.

According to a particular embodiment, the grafted compound is a compound of formula (I).

According to another particular embodiment, the compound of formula (I) is selected from:

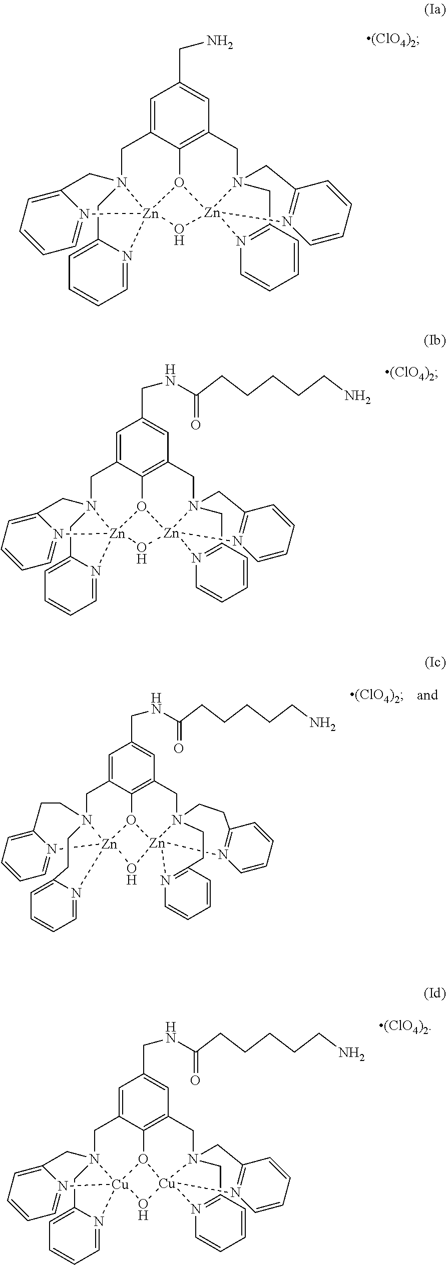

It is to be understood that the present application also describes the compounds of formula (Ia), (Ib), (Ic) and (Id) associated with a counterion different from perchlorate, in particular selected from the anions tosylate, nitrate, sulfate, sulfonate, thiosulfate, halide, hexafluorophosphate, tetraphenylborate and tetrafluoroborate, more particularly from the nitrate, sulfate, halide and carbonate anions. Moreover, the salt used for generating the counterion may in particular be a zinc salt, a copper salt, a manganese salt, a cobalt salt, a nickel salt or an iron salt, more particularly a zinc salt, in particular zinc perchlorate, zinc nitrate, zinc sulfate, a zinc halide or a zinc carbonate.

According to a particular embodiment, the compound is a compound of formula:

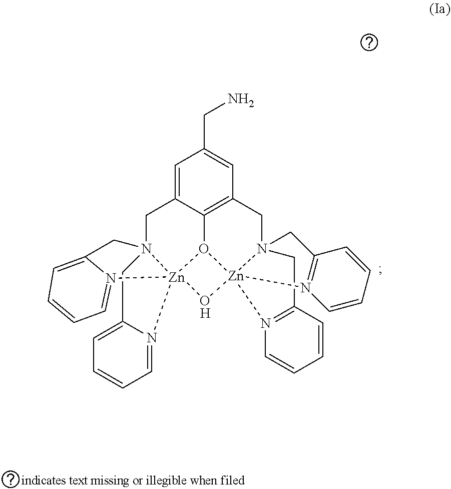

it being understood that the counterion may be different from perchlorate, and in particular selected from the anions tosylate, nitrate, sulfate, sulfonate, thiosulfate, halide, hexafluorophosphate, tetraphenylborate and tetrafluoroborate, more particularly from the nitrate, sulfate, halide and carbonate anions. According to a particular embodiment, the counterion is a perchlorate anion. Moreover, the salt used for generating the counterion may in particular be a zinc salt, a copper salt, a manganese salt, a cobalt salt, a nickel salt or an iron salt, more particularly a zinc salt, in particular zinc perchlorate, zinc nitrate, zinc sulfate, a zinc halide or a zinc carbonate.

The compounds of formula (I) or (II) may be prepared according to the embodiments presented in application PCT/FR2012/050610.

The solid support used in the context of the invention may in particular be a microtiter plate, a sheet, a cone, a tube, a well, a bead, a particle, a strip, a film, a thread, a screw or a needle. Generally, the solid support according to the invention may be used for making any type of material of variable geometry and porosity.

In a particular embodiment, the solid support is a polymeric, metallic or ceramic support, functionalized by means of a compound of formula (I) or (II). According to a particular embodiment, the support is a polymeric support, in particular a support made of poly(vinyl chloride) (or PVC), poly(ethylene terephthalate) (or PET), or polystyrene (or PS).

In the context of the invention, “grafted to its surface” means, in reference to the grafting of the compound of formula (I) or (II) on the support, a covalent bond between the support and the compound of formula (I) or (II). As described below, the compound of formula (I) or (II) may be bound covalently to the support, directly or indirectly. In the case of an indirect covalent bond, the compound of formula (I) or (II) is bound covalently to a reactive function supplied to the surface of the support by a prefunctionalization agent, which has also been bound covalently to the support.

The solid support may be pretreated before grafting the compound of formula (I) or (II). The pretreatment may in particular have the aim of breaking undesirable functions on the surface of the support, in particular ester functions, increasing the density of desired functions, and/or prefunctionalizing the support by making it more electrophilic. In the context of the present invention, when the support is prefunctionalized, said prefunctionalization is carried out covalently by means of a prefunctionalization agent. According to a particular embodiment, the support used is prefunctionalized. The support may thus be prefunctionalized by means of glutaraldehyde, N-hydroxysuccinimide (NHS) or N-oxysuccinimide (NOS), for example. According to a representative embodiment in which the support is in particular made of PET, pretreatment may in particular comprise hydrolysis of the ester functions present on the surface of the support, increase in the density of COOH functions, in particular by oxidation of the support, in particular by means of potassium permanganate, and increase of the electrophilic character of this surface by modification with an attracting group and a good leaving group such as the NHS group. According to a particular embodiment, the PET used (Oxidized PET) comprises a density of COOH functions on its surface comprised between 1×10¹² COOH/cm² and 1×10¹⁸ COOH/cm², in particular between 1×10¹³ and 1×10¹⁷, more particularly between 1.2×10¹⁵ and 1.2×10¹⁷, as measured by means of oxidized toluidine blue (TBO). According to one variant, the support is made of PET and comprises between 5×10¹⁵ and 5×10¹⁶ COOH functions/cm² on its surface before prefunctionalization, more particularly 1.26×10¹⁶±10% COOH functions/cm², in particular before prefunctionalization thereof by means of an NHS group.

Thus, according to one embodiment, the support is a support made of PET, in particular of prefunctionalized PET, in particular by means of NHS functions. According to a particular embodiment, the prefunctionalized PET comprises a density of COOH functions on its surface comprised between 1×10¹³ COOH/cm² and 1×10¹⁸ COOH/cm², in particular between 1×10¹⁴ and 1×10¹⁷, more particularly between 8.1×10¹⁴ and 8.2×10¹⁶. According to one variant, the support made of prefunctionalized PET comprises between 5×10¹⁵ and 1×10¹⁶ COOH functions/cm² on its surface, more particularly 8.18×10¹⁵±10% COOH functions/cm².

According to another particular embodiment, the support is made of PVC, in particular of prefunctionalized PVC, in particular by means of glutaraldehyde.

According to a particular embodiment, the solid support is a support made of PS, in particular of prefunctionalized PS, in particular by means of N-oxysuccinimide (NOS) functions. In particular, the density of NOS functions of the support made of PS thus prefunctionalized may be comprised between 10¹³ and 10¹⁶ NOS/cm², in particular between 10¹⁴ and 10¹⁵ NOS/cm², the density being more particularly of about 68×10¹⁴ NOS/cm².

The compound of formula (I) or (II) is immobilized covalently on the solid support, optionally prefunctionalized, by introducing said support into a solution comprising the compound of formula (I) or (II) to be grafted. According to the embodiment in which the support is prefunctionalized, the prefunctionalization agent is bound covalently to the support. According to a particular embodiment, the solution comprises between 10⁻⁵ and 10⁻² M of compound of formula (I) or (II), in particular between 10⁻⁴ and 5×10⁻² M, more particularly between 5×10⁻⁴ and 5×10⁻³ M. The compound of formula (I) or (II) may in particular be at a concentration of about 10⁻³ M in the solution. The immobilization time may vary widely. However, according to a particular embodiment, the support is brought into contact with the compound of formula (I) or (II) for between 1 h and 72 h, in particular between 5 h and 48 h, more particularly between 10 h and 24 h, and even more particularly for about 16 h.

The invention further relates to a support comprising, grafted on its surface, a compound of formula (I) or (II), said support being included in a kit intended for the detection or characterization of cellular microvesicles. Such a kit may advantageously be used, in particular in the medical analytical laboratory, for the diagnosis of diseases of interest. The kit according to the invention comprises a support according to the invention, and may optionally comprise any means usable for carrying out the method according to the invention. Thus, the kit may in particular comprise means for carrying out a preliminary purification of the microvesicles, if this should prove useful or necessary. The kit may also comprise buffers usable during execution of the method according to the invention, in particular buffers for suspending the microvesicles, washing buffers, or buffers for storage. Means for detecting or quantifying one or more markers that may be present on or in the microvesicles, in particular means for detecting or quantifying protein markers or nucleic acids may be included in the kit. To this end, the kit according to the invention may in particular comprise:
a support as described above,
means for detecting or quantifying one or more markers, and
optionally, buffers usable for carrying out the method according to the invention.

Moreover, the kit may also comprise the means for detecting or quantifying a normalization marker, in particular a normalization marker selected from annexin-A5 and beta-actin.

Thus, according to a particular embodiment, the kit according to the invention may comprise:
a support as described above,
means for detecting or quantifying one or more markers,
means for detecting or quantifying one or more normalization markers, and
optionally, buffers usable for carrying out the method according to the invention.

According to another particular embodiment, the invention relates to a kit comprising:
a support as described above,
means for detecting or quantifying podocalyxin,
means for detecting or quantifying a normalization marker, in particular a normalization marker selected from annexin-A5 and beta-actin, more particularly annexin-A5, and
optionally, buffers usable for carrying out the method according to the invention.

According to another particular embodiment, the invention relates to a kit comprising:
a support as described above,
means for detecting or quantifying alpha-synuclein,
means for detecting or quantifying a normalization marker, in particular a normalization marker selected from annexin-A5 and beta-actin, more particularly annexin-A5, and
optionally, buffers usable for carrying out the method according to the invention.

According to another particular embodiment, the invention relates to a kit comprising:
a support as described above,
means for detecting or quantifying CD41,
   means for detecting or quantifying a normalization marker, in particular a normalization marker selected from annexin-A5 and beta-actin, more particularly annexin-A5, and
optionally, buffers usable for carrying out the method according to the invention.

“Means for detecting or quantifying” means any means known by a person skilled in the art for detecting or quantifying a marker. Of course, the means employed will depend on the nature of the marker, a protein marker in particular being detectable by immunologic techniques (in particular ELISA and Western blot) and nucleic acid markers being detectable in particular by means of specific amplification techniques, which may be qualitative or quantitative (in particular PCR/qPCR or RT-PCR/RT-qPCR, or sequencing). Other means include chromogenic assays, depending on the nature of the biomarker being detected.

Moreover, the kit according to the invention may comprise a leaflet providing the user with instructions for carrying out the method according to the invention by means of the kit.

The invention therefore also relates to the use of the support according to the invention for capturing microvesicles present in a sample of biological fluid from a subject. The data presented by the inventors show that it is possible to perform detection of biomarkers in various types of biological samples, in particular in urine or blood, more particularly in plasma, by means of the method presented in the present application. The method according to the invention is therefore capable of providing detection of microvesicles in an extensive panel of samples of biological fluids. The biological fluid may be, in particular, a sample of blood, serum, plasma, saliva, tears, urine, lymphatic fluid, cerebrospinal fluid, or semen. The subject is a mammal, in particular a human being, of any age, sex or condition. According to a particular embodiment, the subject is an individual with a suspected pathological state, in particular on the basis of bioassays or medical consultations carried out beforehand. In another embodiment, the subject has not undergone a bioassay or prior medical consultation.

The invention therefore relates to a method for capturing microvesicles, comprising bringing a sample of biological fluid that may contain said vesicles into contact with a support according to the invention. The microvesicles thus captured may then be characterized. According to one embodiment, prior to capture with the support according to the invention, the microvesicles are first purified or isolated from the sample of biological fluid by procedures known by a person skilled in the art. However, the experimental data presented hereunder show that the microvesicles may advantageously be captured directly in a sample of biological fluid by means of the support according to the invention. Thus, in a particular embodiment, the microvesicles are captured directly from the sample of biological fluid, in particular directly from a sample of blood, serum, plasma, saliva, tears, urine, lymphatic fluid, cerebrospinal fluid, or semen, more particularly from plasma or urine.

According to a particular embodiment, characterization of the microvesicles captured allows diagnosis of a disease, evaluation of the risk of developing a disease, prognosis of a disease, differential diagnosis of a disease, monitoring the progression of a disease, or monitoring the efficacy of a therapeutic treatment of a disease. As mentioned above, the composition of the cellular microvesicles, in particular the lipid and protein composition, and the contents of the cellular microvesicles (e.g. proteins and genetic material, in particular RNA or mitochondrial DNA), may vary depending on the type of cell from which they originated, and the state of the cell. The microvesicles may therefore allow detection of the state of the cells from which they originated, and may therefore represent a tool of choice for the early detection of a pathological state. Thus, according to one aspect, the invention relates to the use of a support according to the invention in a method for diagnosis, differential diagnosis, evaluation of the risk, prognosis, monitoring of the progression or monitoring of the efficacy of a therapeutic treatment of various diseases, in particular thrombotic, inflammatory and/or metabolic, or of cardiovascular or neurovascular diseases or accidents, or of diseases such as diabetes, cancer, Alzheimer's disease, Parkinson's disease or any other diseases.

The invention therefore also relates to a method of diagnosis, differential diagnosis, risk evaluation, prognosis, monitoring of the progression or monitoring of the efficacy of a therapeutic treatment of various diseases, in particular thrombotic, inflammatory and/or metabolic, or of cardiovascular or neurovascular diseases or accidents, or of diseases such as diabetes, cancer, Alzheimer's disease, Parkinson's disease or any other disease. We may thus mention diabetic nephropathy, diabetic neuropathy, diabetic retinopathy, multiple sclerosis, vasospasm after rupture of an aneurysm, Parkinson's disease or derivatives thereof. Depending on the specific aim, the result of the characterization of the sample tested will be compared against the result of the characterization carried out on a sample of a control biological fluid.

Advantageously, the control biological fluid is a biological fluid identical to that assayed, but coming from a subject considered to be healthy. Alternatively, the control biological fluid is from the same individual as the biological fluid assayed, but results from a previous sample-taking. A control according to this alternative may allow monitoring of the progression of the disease or of the treatment thereof.

The characterization of the microvesicles may be carried out by any means known by a person skilled in the art, the nature of the characterization marker being taken into account, as was mentioned above. Thus, in the case of a protein marker, immunological techniques may be employed, using specific antibodies of said protein marker. We may mention in particular the ELISA or Western blot techniques. The detection and characterization of nucleic acids, in particular of mitochondrial DNA or of RNA (in particular messenger RNA or microRNA) will comprise the use of methods of detection of specific nucleic acids such as PCR/qPCR, RT-PCR/RT-qPCR, and sequencing. Other assays, such as chromogenic assays, may be used depending on the nature of the biomarker to be detected. Advantageously, normalization may be carried out based on quantification of a marker present on or in the microvesicles. The normalization marker may in particular be selected from annexin-A5 and beta-actin. For example, the experimental data presented below show that the markers podocalyxin, alpha-synuclein and CD41 may be used for the diagnosis of specific diseases. According to a particular embodiment, quantification of these markers will be carried out on a relative basis by parallel quantification of a normalization marker such as annexin-A5 and beta-actin. Thus, according to a particular embodiment, the method according to the invention comprises detection of the amount of a biomarker present in or on the microvesicles captured and detection of the amount of a normalization marker. The quantities normalized are then compared for determining a diagnosis, a differential diagnosis, for evaluating the risk, the prognosis, monitoring of the progression or monitoring of the efficacy of a therapeutic treatment of various diseases.

According to a second aspect, the invention relates to the use of the biomarkers allowing detection of the presence or absence of a specific disease. The characterization biomarker will be selected in relation to the disease of interest.

According to one embodiment, the invention relates to a method of diagnosis, in particular of early diagnosis, of a nephropathy in a subject, comprising detection of the presence or absence of podocalyxin, or measurement of the level of podocalyxin in a sample of biological fluid from said subject. According to a particular embodiment, the method comprises measurement of the ratio of podocalyxin to the normalization marker (in particular annexin-A5 or beta-actin). More particularly, the method according to the invention may in particular comprise detection of the presence or absence of cellular microvesicles comprising the marker podocalyxin, according to the embodiments described above comprising the use of a support according to the invention. The invention also relates to the evaluation of the risk of development of a nephropathy, prognosis of a nephropathy, monitoring the progression of a nephropathy, or monitoring the efficacy of a therapeutic treatment of a nephropathy comprising detection of the presence or absence of podocalyxin, or measurement of the level of podocalyxin in a sample of biological fluid from a subject, in particular of podocalyxin comprised in cellular microvesicles characterized according to the embodiments disclosed above. According to a particular embodiment, the method according to the invention comprises quantifying the podocalyxin/annexin-A5 ratio or podocalyxin/beta-actin ratio, more particularly the podocalyxin/annexin-A5 ratio, for detecting nephropathic complications in the microvesicles derived from a sample of biological fluid from a patient, more particularly a urine sample. This ratio, when it is statistically higher than that calculated in a healthy subject, is indicative of a nephropathy. Moreover, if this ratio increases or decreases relative to the ratio calculated in the past for the same patient, it is evidence of progression or of regression of the nephropathy, respectively.

Moreover, the invention relates to a method of diagnosis of Parkinson's disease in a subject, comprising detection of the presence or absence of alpha-synuclein, or measurement of the level of alpha-synuclein in a sample of biological fluid from said subject. According to a particular embodiment, the method comprises measurement of the ratio of alpha-synuclein to a normalization marker (in particular annexin-A5 or beta-actin). More particularly, the method according to the invention may in particular comprise detection of the presence or absence of cellular microvesicles comprising the marker alpha-synuclein, according to the embodiments described above comprising the use of a support according to the invention. The invention also relates to evaluation of the risk of developing Parkinson's disease, prognosis of a Parkinson's disease, monitoring the progression of a Parkinson's disease, or monitoring the efficacy of a therapeutic treatment of Parkinson's disease, comprising detection of the presence or absence of alpha-synuclein, or measurement of the level of alpha-synuclein in a sample of biological fluid from a subject, in particular of alpha-synuclein comprised in cellular microvesicles or in the membranes of cellular microvesicles characterized according to the embodiments disclosed above. According to a particular embodiment, the method according to the invention comprises quantifying the alpha-synuclein/annexin-A5 ratio or alpha-synuclein/beta-actin ratio, more particularly the alpha-synuclein/annexin-A5 ratio, for detecting these neurological disorders (for example, Parkinson's disease) in the microvesicles derived from a sample of biological fluid from a patient. When this ratio is statistically higher than that calculated for a healthy subject, it is indicative of a Parkinson's disease. Moreover, if this ratio increases or decreases relative to the ratio calculated in the past for the same patient, it is evidence of progression or of regression of Parkinson's disease, respectively.

The invention further relates to a method of diagnosis, in particular of early diagnosis, of a diabetic nephropathy in a subject. According to a particular embodiment, the method according to the invention comprises detection of the presence or absence of the platelet marker CD41, or measurement of the level of CD41 in a sample of biological fluid from said subject, in particular a sample of urine or of plasma, more particularly a plasma sample. According to a particular embodiment, the method comprises measurement of the ratio of CD41 to a normalization marker (in particular annexin-A5 or beta-actin). More particularly, the method according to the invention may in particular comprise detection of the presence or absence of cellular microvesicles comprising the marker CD41, according to the embodiments described above comprising the use of a support according to the invention. According to a particular embodiment, the method according to the invention comprises quantifying the CD41/annexin-A5 ratio or CD41/beta-actin ratio, more particularly the CD41/annexin-A5 ratio, for detecting the nephropathic complications of diabetes in the microvesicles derived from a sample of biological fluid from a patient, in particular a sample of urine or of plasma. When this ratio is statistically higher than that calculated for a healthy subject, it is indicative of a diabetic nephropathy. Moreover, if this ratio increases or decreases relative to the ratio calculated in the past for the same patient, it is evidence of progression or of regression of the diabetic nephropathy, respectively.

Detection of the stage of nephropathy may in particular comprise comparing the result of characterization obtained from the test sample of biological fluid with the result of characterization of one or more control samples characteristic of different stages of the disease (in particular obtained from diabetic patients with nephropathy at the normoalbuminuric stage (Normo), microalbuminuric stage (Micro) or macroalbuminuric stage (Macro), these stages corresponding to the severity of the disease, the Macro stage being the most advanced). According to a particular embodiment, the method according to the invention thus allows monitoring and early detection of diabetes and of the potential complications thereof, in particular the renal or neuropathic complications thereof.

The invention will now be illustrated by the following nonlimiting examples.

### EXAMPLES

### Abbreviations

DCM: dichloromethane

DMSO: dimethylsulfoxide

Eq: equivalent

EtOAc: ethyl acetate

MES: 2-(N-morpholino)ethanesulfonic acid

MilliQ: water purified with a Milli-Q water purification system

MV: microvesicle

NHS: N-hydroxysuccinimide

NOS: N-oxysuccinimide

Cplx: complex

PVC: poly(vinyl chloride)

PS: phosphatidylserine

Pht: phthalimide

MeCN: acetonitrile

XPS: X-ray photoelectron spectroscopy

EDA: ethylenediamine

HUVEC: Human Umbilical Vein Endothelial Cells

The complexes used in this experimental section were synthesized according to the embodiments presented in application PCT/FR2012/050610, in particular the complexes C1, C2 and C4 of formulas:

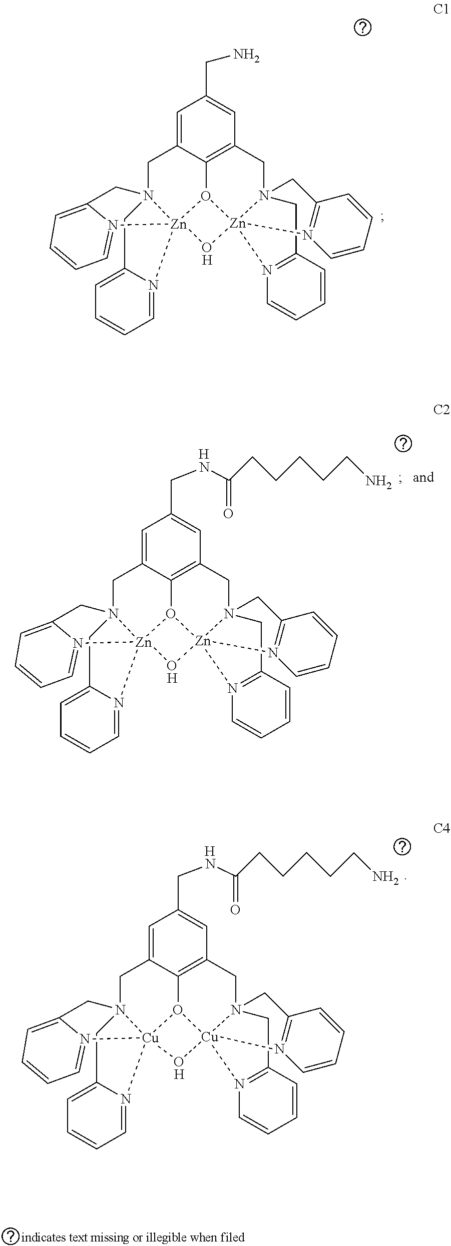

### Example 1: Functionalization of the PET Support and Grafting of the Complexes on the Surface

Polyethylene terephthalate (PET) was selected for covalent grafting of the complexes, this grafting is taking place owing to their primary amine function reacting with the carboxylic acids present on the surface.

General Protocol:

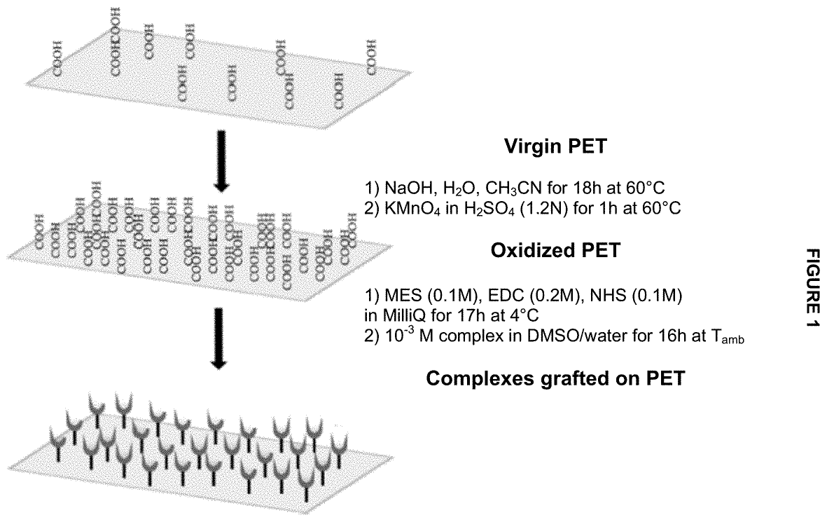

First, functionalization steps (FIG. 1) were used for i) breaking the ester bonds, ii) increasing the density of the COOH functions, and iii) making the carbonyls more electrophilic by means of an attracting group and good leaving group, N-hydroxysuccinimide, in order to react more quickly and with a better yield with the complexes.
1) Virgin PET film (obtained from Goodfellow) was precut into a rectangle of 5 cm by 2 cm in order to obtain 10 small squares of 1 cm². This rectangle was cleaned in a tube (falcon) containing 96% ethanol for 1 h, with sonication. The PET was dried for a few minutes on Kimtech papers,
2) The cleaned rectangle of virgin PET was placed in another falcon containing a solution of sodium hydroxide (NaOH 10⁻³ M in 40 mL MilliQ/MeCN v/v 1/1) for 18 h at 60° C.,
3) Washing with MilliQ water (3 times) and then for 48 h,
4) Oxidation of the PET rectangle in a solution of potassium permanganate (2 g KMnO₄ in 38.4 mL of MilliQ water and 1.6 mL H₂SO₄) for 1 h at 60° C.,
5) Washing with acid (HCl 6M) and then washing with MilliQ water (2 times) and cutting the rectangle into small squares of 1 cm by 1 cm,
6) The squares were put in a tablet container with 2 mL of a solution of (76.7 mg of EDC, 23 mg NHS, 39 mg MES in 2 mL of MilliQ water for one square)
7) Each square is placed in a tablet container containing 2 mL of solution of complex (10⁻³ M in 0.2 mL of DMSO and 1.8 mL MilliQ) for 16 h at room temperature.
8) Rinsing with MilliQ water (3 times, with sonication) and washing for 48 h.

“Control” samples required for characterization of the materials were prepared.

These are samples of virgin PET without hydrolysis or oxidation and without EDC/NHS activation. Steps 1), 7) and 8) are carried out but without steps 2), 3), 4), 5) and 6).

Characterization of the Materials

Quantification of the Density of Carboxylic Acid on the Surface for all the Functionalization Steps:

The method used for quantifying the density of COOH groups present on the surface of the polymer is the method using Toluidine Blue O, as this cationic chemical reagent reacts on the surface with the COO⁻ groups. Indeed, a basic pH (for example pH=10) is necessary for deprotonating the carboxylic acid groups so that they react with TBO to form a noncovalent bond. For resorbing the TBO fixed on the surface of the material, acetic acid is added to reprotonate the COO⁻ groups and thus break the ionic bond [COO⁻. . . TBO].

A calibration curve is constructed beforehand, with different concentrations of TBO.

The optical density at 630 nm read for the samples was reported, and the calibration curve enabled us to find the surface concentration of COOH.

From the calibration curve, in the case of virgin PET (PET as delivered by the supplier and washed with ethanol and then dried), we obtain a density of COOH functions of 1.5 μmol/mm².

In the same way, the surface density of COOH functions could be evaluated on the PET surfaces at the different steps of the treatment. We get:
28 μmol/mm² for hydrolyzed PET
210 μmol/mm² for oxidized PET
136 μmol/mm² for PET-NHS
50 μmol/mm² for PET+C1.

Determination of Atomic Percentages on the Surface of PET at the Different Steps of the Treatment by Photoelectron Spectroscopy

These analyses were carried out with the “VG ESCALAB 220i-XL” spectrometer. Atomic quantification of the Si, S, C, N, O or Zn atoms was carried out by X-ray photoelectron spectroscopy (XPS), in the solvent DMSO/H₂O 10/90.

TABLE 1
PET NHS activated*

| Atom | at % |
|---|---|
| Si | 0.72 ± 0.13 |
| S | 0.28 ± 0.14 |
| C | 70.85 ± 0.13 |
| N | 2.19 ± 0.28 |
| O | 25.98 ± 0.52 |

*N-hydroxysuccinimide group on hydrolyzed and oxidized PET to increase the COOH number and thus increase the number of complexes per unit of surface area

TABLE 2
PET + C1 in DMSO

| Atom | at % |
|---|---|
| Si | 0.34 ± 0.07 |
| S | 0.21 ± 0.04 |
| C | 73.39 ± 0.25 |
| N | 3.76 ± 0.11 |
| O | 20.83 ± 0.30 |
| Zn | 1.48 ± 0.04 |

The quantifications of the atomic percentages make it possible to state that the complex has indeed been immobilized on the surface of the PET. Indeed, relative to the activated PET that does not contain any metal, PET+C1 contains an atomic percentage of Zn. Moreover, we may note an increase in the percentage of nitrogen expected theoretically since the complex contains 6 nitrogen atoms at the level of the ligand. This result is supported by the ratios such as COO/C—O which changes from 0.93 (PET-NHS) to 0.65-0.68 (PET+C1) and NCO/COO which changes from 0.06 (PET-NHS) to 0.17-0.25 (PET+C1).

XPS Analysis of Functionalized Vs Unfunctionalized Supports, and/or Grafted with the Complexes C1, C2 or C4

These analyses were carried out with the “VG ESCALAB 220i-XL” spectrometer. 9 samples were prepared for X-ray photoelectron spectroscopy analysis: Virgin PET cleaned (Smp1), oxidized PET (Smp2), PET-NHS or activated PET (i.e. PET hydrolyzed, oxidized and then activated with EDC/NHS; Smp3), activated PET+C1 (Smp4), activated PET+C2 (Smp5), activated PET+C4 (Smp6), Virgin PET+C1 (Smp7), Virgin PET+C2 (Smp8) and Virgin PET+C4 (Smp9).

The high-resolution spectra O1s obtained by XPS show that the complexes are grafted on the surface of the material based on the appearance of peaks for “Zn—O” and “Cu—O” at 530.5-530.7 eV starting from sample 4. At the level of the area ratios Zn—O (or Cu—O)/C═O, the value is 0.15 for Smp4, 0.25 for Smp5, 0.11 for Smp6 and the latter decreases without the activation step, 0.08 for Smp7, 0.15 for Smp8 and 0.14 for Smp9. These results can be explained by the fact that without activation (Smp 7, 8 and 9), the complexes are adsorbed and not grafted covalently to the surface of the support. Thus, after several rinsings, the complexes are removed and are almost no longer detected.

TABLE 3

| at % | | | | | | |
|---|---|---|---|---|---|---|
| Materials | Si | S | C | N | O | Zn or Cu |
| Smp1 | 1.23 ± 1.24 | | 70.57 ± 1.46 | 1.77 ± 0.09 | 26.44 ± 0.28 | |
| Smp2 | 0.41 ± 0.22 | | 71.85 ± 0.15 | 2.34 ± 0.19 | 25.41 ± 0.2 | |
| Smp3 | 1.49 ± 0.42 | | 69.55 ± 0.88 | 2.45 ± 0.21 | 26.49 ± 0.35 | |
| Smp4 | 0.7 ± 0.03 | 0.06 ± 0.03 | 71.92 ± 0.34 | 4.2 ± 0.22 | 22.18 ± 0.52 | 0.93 ± 0.03 |
| Smp5 | 0.51 ± 0.21 | 0.18 ± 0.03 | 72.27 ± 0.27 | 4.62 ± 0.43 | 21.44 ± 0.51 | 0.97 ± 0.1 |
| Smp6 | 0.47 ± 0.17 | 0.15 ± 0.02 | 72.17 ± 0.33 | 3.37 ± 0.19 | 23.3 ± 0.22 | 0.53 ± 0.03 |
| Smp7 | 2.01 ± 0.23 | 0.1 ± 0.02 | 74.4 ± 1.1 | 2.46 ± 0.21 | 20.76 ± 1.26 | 0.27 ± 0.02 |
| Smp8 | 2.71 ± 0.57 | 0.25 ± 0.04 | 75.6 ± 0.19 | 1.73 ± 0.19 | 19.43 ± 1.18 | 0.27 ± 0.06 |
| Smp9 | 1.49 ± 0.26 | 0.18 ± 0.08 | 71.56 ± 0.42 | 2.34 ± 0.13 | 23.98 ± 0.68 | 0.43 ± 0.09 |

When covalent grafting takes place, a peak at 400 eV (high-resolution spectrum N1s) corresponding to the amide bond N—C═O is easily observed by XPS, which means that the bond between the NH₂ of the complex and the carbonyl of the material is indeed produced. Furthermore, when EDC/NHS activation is not carried out and the complexes are brought into contact with the surface (Smp 7, 8 and 9), a different peak than that at 400 eV is observed. This peak corresponds to the free NH₂ borne by group X of the complex, which could be correlated with adsorption and not with a covalent bond between the complex and the material.

Optimization of the Density of the Complexes Grafted on the Surface

So as to use only the necessary amount of complexes on the surface, to promote better accessibility of the complexes by the phosphatidylserine present on the microvesicles, in a variant, the support may be prepared following the general protocol in which steps 2 and 3 are not carried out or in which steps 2, 3, 4 and 5 are not carried out.

Indeed, by omitting the steps of hydrolysis and/or of oxidation of the PET, it is possible to modulate the quantity of COOH groups on the surface of the PET and therefore modulate the quantity of complexes immobilized on the surface of the PET. In the proposed variant, virgin PETs are cleaned and then functionalized without prior oxidation or without prior oxidation and hydrolysis. With the proposed variant, only the —COOH functions present on the surface of the support are activated/functionalized with a view to the grafting of complexes. Advantageously, as the PET that has not undergone an oxidation step contains a smaller quantity of carboxylic acid groups, the grafting density obtained will therefore be lower (d1)
1.5 μmol/mm² of COOH for virgin PET
28 μmol/mm² of COOH for hydrolyzed PET
210 μmol/mm² of COOH for oxidized PET.

### Example 2: Functionalization of PVC and Grafting of the Complexes

General Protocol:

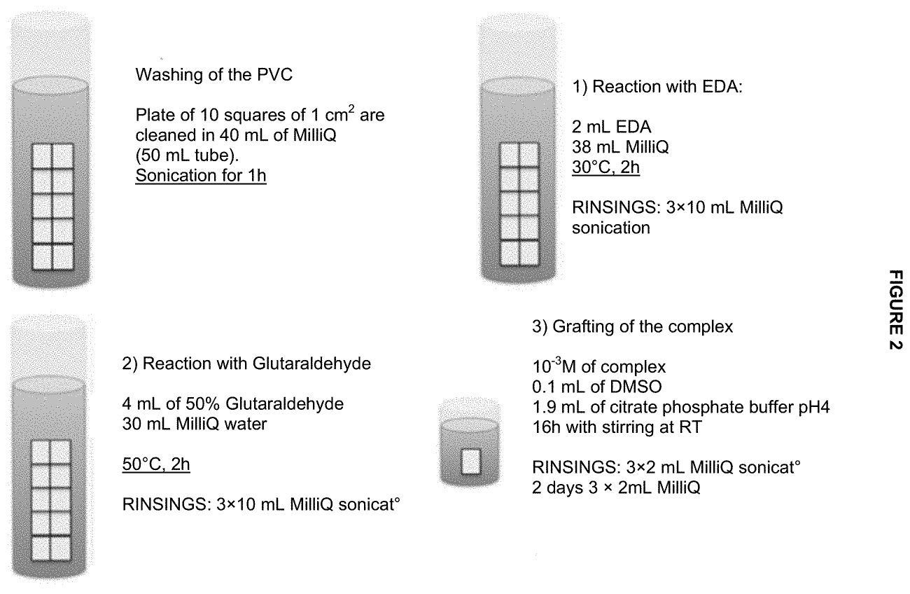

The protocol employed will be described referring to FIG. 2.

1) A PVC plate with 10 squares of 1 cm² was cleaned by sonication for 1 h in 40 mL of MilliQ water in a 50 mL tube. Then the plate was immersed in a solution comprising 2 mL of 97% EDA and 38 mL of MilliQ water, for 2 h at 30° C. Finally, the plate of 10 squares was rinsed 3 times in 10 mL of MilliQ water, sonication being carried out at each rinsing step.

2) The plate was immersed in a solution comprising 4 mL of 50% glutaraldehyde and 30 mL of MilliQ water at pH 9.5, for 2 h at 50° C. Then the plate was rinsed 3 times in 10 mL of MilliQ water, sonication being carried out at each rinsing step.

3) Finally, the complex is grafted by immersing a square from each plate treated according to the above steps in a solution comprising 10⁻³ M of complex, 0.1 mL of DMSO and 1.9 mL of citrate phosphate buffer (0.5 M citric acid, 0.5 M disodium sodium phosphate) at pH4, for 16 h at room temperature with stirring. Then the PVC square was rinsed 3 times in 2 mL of MilliQ water, sonication being carried out at each of these rinsing steps. The treated PVC square was then rinsed 3 times in 2 mL of MilliQ water over 2 days.

Characterization of the Surfaces by Photoelectron Spectroscopy (XPS)

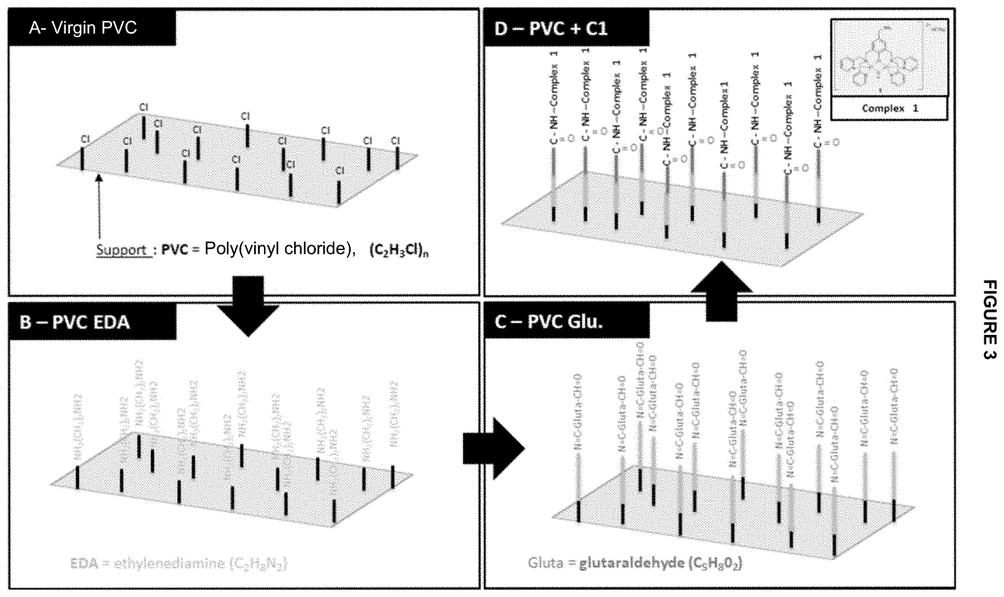

To characterize the surface of the materials, analyses by XPS spectroscopy on PVC were carried out at each step of their functionalization with complex C1. The materials are respectively shown schematically in FIG. 3. Five surface points, 4 corners and 1 center, were studied for each material. These analyses were carried out with the “VG ESCALAB 220i-XL” spectrometer.

Results

The spectra and deconvolutions of the spectra as well as the atomic percentages (Table 3) of the various elements present on the surface of the materials show that, after functionalization with complex C1, zinc (Zn) is present on the surface of the materials at a level of 0.25% for PVC. Zinc being the metal in the composition of complex C1, its presence tells us that complex C1 is fixed on the surface of the materials. The functionalization is therefore effective on PVC. Moreover, the atomic percentages determined at five different points of the surface (4 corners+1 center) show low standard deviations (Table 3), demonstrating uniformity of the treatment and reproducibility of the results. From this, we deduce that the functionalization is uniform on PVC.

### Example 3: Grafting of Complex C1 on a DNA-BIND® Plate

General Protocol

The complexes were immobilized on DNA-BIND® commercial 96-well plates from Costar. This plate is of polystyrene prefunctionalized with N-oxysuccinimide (NOS) functions. This plate contains 68×10¹⁴ NOS/cm². This surface density of NOS is comparable to the density of COOH functions that we had on polyethylene terephthalate (PET) (8.18×10¹⁵ COOH/cm²)

1) A solution is prepared (Vtotal=2 mL) with −1.78 g of Complex −0.2 mL DMSO −1.8 mL of MilliQ water. 200 μL of this solution is deposited in each well (10 wells). The grafting reaction is carried out for 16 h at room temperature away from the light.

2) After this reaction time, the solution is removed from the wells and the wells are rinsed by 3 flushes with MilliQ water. Each well is then incubated with fresh MilliQ water and is rinsed 3 times/day for 48 h.

Characterization of the Surfaces by Photoelectron Spectroscopy

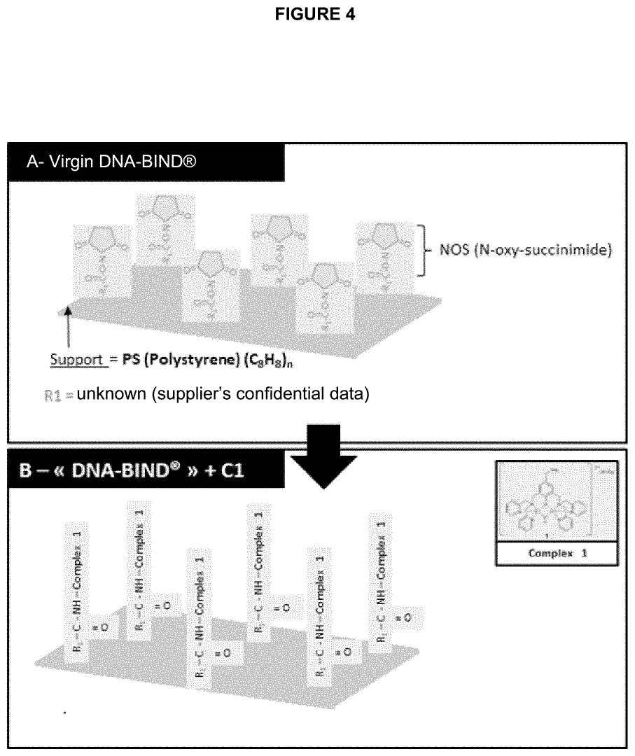

To characterize the surface of the materials, XPS spectroscopy analyses on “DNA-BIND®” were carried out at each step of their functionalization with complex C1. The materials are respectively shown schematically in FIG. 4. Five surface points, 4 corners and 1 center, were studied for each material. These analyses were carried out with the “VG ESCALAB 220i-XL” spectrometer.

Results

The spectra and deconvolutions of the spectra as well as the atomic percentages (Table 4) of the various elements present on the surface of the materials show that, after functionalization with complex C1, zinc (Zn) is present on the surface of the materials, at a level of 0.41% for “DNA-BIND®”. As zinc is the metal in the composition of complex C1, its presence tells us that complex 1 is immobilized on the surface of the materials. The functionalization is therefore effective on DNA-BIND®. Moreover, the atomic percentages determined at five different points of the surface (4 corners+1 center) show low standard deviations (Table 4), demonstrating uniformity of the treatment and reproducibility of the results. From this it is deduced that the functionalization is uniform on the two materials: PVC and DNA-BIND®.

TABLE 4

| | Materials | | | | | |
|---|---|---|---|---|---|---|
| | | | | | Virgin | DNA- |
| | Virgin | PVC | | | DNA- | BIND ® + |
| at % | PVC | EDA | PVCGlu. | PVC+ C1 | BIND ® | C1 |
| C | 74.06 ± 0.03 | 73.74 ± 0.51 | 74.90 ± 0.34 | 78.87 ± 1.40 | 96.52 ± 0.26 | 91.59 ± 2.25 |
| N | 0.51 ± 0.23 | 0.81 ± 0.29 | 0.67 ± 0.07 | 2.08 ± 0.20 | 0.78 ± 0.09 | 1.73 ± 0.09 |
| O | 6.58 ± 0.12 | 6.27 ± 0.49 | 8.43 ± 0.45 | 8.69 ± 0.21 | 2.60 ± 0.26 | 5.69 ± 1.81 |
| Cl | 18.64 ± 0.25 | 18.89 ± 1.07 | 15.7 ± 0.25 | 9.46 ± 1.15 | / | / |
| Zn | / | / | / | 0.25 ± 0.05 | / | 0.41 ± 0.03 |
| Si | 0.18 ± 0.03 | 0.18 ± 0.09 | 0.18 ± 0.05 | 0.50 ± 0.12 | 0.16 ± 0.09 | 0.16 ± 0.05 |
| S | 0.10 | 0.18 ± 0.16 | 0.07 | 0.12 ± 0.04 | / | 0.28 ± 0.21 |
| Na | / | / | / | / | / | 0.44 ± 0.28 |
| Sn | / | 0.15 | 0.17 | 0.11 | / | / |

Atomic percentages of the elements present on the surface of the materials PVC and “DNA-BIND ®” obtained by XPS after each step of their functionalization with complex 1 at five points on the surface (4 corners + 1 center) as well as the corresponding standard deviations.

### Example 4: Search for Markers Carried by the Microvesicles Following their Capture by the Kit DNA-BIND® Polystyrene Grafted with Complex C1

Materials and Methods

a/ Sampling of Biological Fluids from Diabetic Patients

Samples of urine and plasma were obtained from diabetic patients in the diabetology unit of Pr Vincent Rigalleau. All the patients signed a free and informed consent that was explained in accordance with the Declaration of Helsinki and was approved by the ethics committee. The clinical picture, including the level of albuminuria in the urine, is established for each diabetic patient. The patients were divided into three groups: Normoalbuminuric patients or Normo (stage 1 of the complications of the disease), Microalbuminuric patients or Micro (stage 2) and Macroalbuminuric patients or Macro (stage 3). Urine samples were also collected from healthy donors, with no known nephropathic complication. All the samples are stored at −80° C. until used.

b/ Cell model: Human Umbilical Vein Endothelial Cells, HUVECs

The endothelial cells, Human Umbilical Vein Endothelial Cells (HUVECs) (Promocell, C-12208) are cultured in Endothelial Cell Growth Medium 2 Kit (Promocell, C-22111) consisting of a basal medium and a mixture of growth factors without antibiotics.

c/ Isolation of Model Microvesicles from Cell Culture (Human Umbilical Vein Endothelial Cells or HUVECs)

For activation of the production of model microvesicles, the HUVECs are rinsed twice with PBS buffer and are stimulated for 24 h in culture medium with 100 ng/mL of TNF-alpha (Peprotech, ref. 300-01A). The culture media of the cells are recovered after 24 h of stimulation and are centrifuged in a 50 mL tube at 12000 g for 2 min at 4° C. in order to remove the cells, cellular debris and apoptotic bodies. The supernatants are transferred to clean tubes and centrifuged at 20000 g for 90 min at 4° C. in order to sediment the microvesicles. The pellet of model microvesicles obtained is washed by resuspension in 1.5 mL of cold PBS buffer, transferred to a 1.5 mL tube and then centrifuged at 20000 g for 90 min at 4° C. This washing step is repeated once. The pellet of model microvesicles is then resuspended depending on the size of the pellet in cold PBS (100 to 500 μL). The amount of corresponding proteins is determined by absorbance at 280 nm using a nanodrop spectrophotometer. The sample of model microvesicles is stored at −80° C. until used.

d/ Purification of Microvesicles from Human Urine Samples

The urine from healthy donors and from patients is thawed and the microvesicles are purified by differential centrifugations: a first centrifugation is carried out at 12000 g for 2 min at 4° C. in order to remove the cells, cellular debris and apoptotic bodies. The supernatant is centrifuged at 20000 g for 90 min at 4° C. in order to sediment the microvesicles. The pellet of microvesicles obtained is washed by resuspension in 1.5 mL of cold PBS buffer, transferred to a 1.5 mL tube and then centrifuged at 20000 g for 90 min at 4° C. This washing step is repeated once. The sample is stored at −80° C. until used.

e/ Purification of Microvesicles from Human Blood Samples

The blood collected in a sodium citrate tube is subjected to a first centrifugation at 1500 g. The upper phase corresponding to the plasma is carefully removed and is centrifuged at 12000 g for 2 minutes in order to remove the platelets. The supernatant corresponding to the platelet-free plasma, PFP, is then centrifuged at 20000 g at 4° C. in order to sediment the microvesicles. The pellet of microvesicles obtained is washed by resuspension in 1.5 mL of cold PBS buffer, transferred to a 1.5 mL tube and then centrifuged at 20000 g for 90 min at 4° C. This washing step is repeated once. The sample is stored at −80° C. until used.

f/ Detection of Protein Biomarkers

Enzymatic Method: The model microvesicles produced by activations of the HUVEC cells have on their surface plasminogen activators, which activate plasminogen (present on the surface of the microvesicles) into plasmin; the activity of this plasm in can be detected using a chromogenic substrate, methylmalonyl-hydroxypropyl-arginyl-paranitroanilide, CBS0065. The chromophore is cleaved in the presence of plasmin into a yellow colored product, whose absorbance can be measured using a spectrophotometer at a wavelength of 450 nm. The microvesicles resuspended in PBS buffer after isolation from HUVECs are deposited in the wells of the 96-well polystyrene plate (DNA-BIND® Costar) grafted with the dinuclear metal complex I, called complex I. A total amount of 2.5 μg of microvesicles (determined by the A280 nm nanodrop technique) in 25 μL is incubated for 1 h at room temperature. After three rinsings, the enzymatic activity of each well is determined. Certain wells are not rinsed and represent the control 100% of enzymatic activity. First, a standard range is obtained by serial dilution with the following concentrations of plasminogen activator in IU/mL: 0, 0.00078125, 0.0015625, 0.003125, 0.00625, 0.0125, 0.025 and 0.05. Secondly, a plasminogen solution at 4 μM and a solution of the chromogenic substrate CBS0065 at 3 mM are prepared and then mixed volume by volume. A volume of 25 μL of this mixture is added to each well. The plate is covered with cling film and placed in the spectrophotometer at 37° C. for 18 h for reading the absorbance simultaneously at 405 nm and at 450 nm in each well. This kinetic study enables us to calculate the average rate of appearance of the product, reflecting the enzymatic activity of each well; it is expressed in mOD/min.

g/ Western Blot

The first step consists of lysis of the microvesicles using RIPA buffer+protease inhibitors and of phosphatases (990 μL of RIPA buffer+10 μL of the cocktail of protease inhibitors and of phosphatases inhibitors (100λ)). Add a volume of RIPA buffer+inhibitors as a function of the size of the pellet of microvesicles obtained in the last step of isolation before adding PBS (volume of RIPA about 4× greater than the volume of the pellet). The pellet is resuspended by pipetting up and down and is left in ice for 15 minutes. Then the lysate is centrifuged at 10000×g for 10 minutes at 4° C. to remove the debris. The supernatant is transferred to a clean tube. The amount of proteins present in the lysate of microvesicles is measured by the BiCinchoninic acid Assay (BCA) technique. The proteins are then denatured using the buffer Laemmli 4X+reducing agent (DTT, dithiothreitol). The denatured lysate can be stored at −80° C. before use. The denatured lysates of microvesicles are deposited on acrylamide gel in SDS-PAGE denaturing conditions at a concentration gradient of 4-12%. Migration is carried out at a constant voltage of 120 V for about 1.5 h (until the migration front reaches the end of the bottom of the gel). The gel is transferred onto a PVDF membrane. After saturation of the membrane for 1 h at room temperature with a 5% milk solution in TBS-Tween 0.1% buffer, the 3 primary antibodies diluted in the 5% milk solution in TBS-Tween 0.1% are incubated overnight at 4° C. The following dilutions are used: antibody against podocalyxin (Santacruz, Sc-23904) 1/2000 or against CD41 (Novus, MAB 7616) at 1/1000; plus antibody against beta-actin (SIGMA, A1978-100UL) 1/10000; plus antibody against annexin-A5 (SIGMA, A8604-100UL) 1/2000. After two washing operations in TBS-Tween 0.1% buffer, the secondary antibodies coupled to the enzyme horseradish peroxidase, HRP, and at a dilution of 1/5000, are incubated for 1 h at room temperature in the 5% milk solution in TBS-Tween 0.1%. Detection by chemiluminescence is carried out after two washing operations in TBS-tween 0.1% buffer. The signal is captured by a CCD camera and then the intensity of the latter is analyzed using the free software ImageJ.

h/ Statistical Analysis

For validation of the biomarker in the microvesicles obtained from the urine of diabetic patients, statistical analysis of the Western blot results was performed with the GraphPad Prism software version 7.

i/ ELISA

The microvesicles resuspended in PBS buffer after isolation from urine or plasma from patients or from healthy individuals are deposited in the wells of the 96-well polystyrene plate (DNA-BIND® Costar) grafted with the dinuclear metal complex I, called complex I. Incubation overnight at 4° C. with gentle horizontal agitation was observed. After washing twice in PBS, the saturated solution consisting of 5% of “bovine serum albumin” (BSA) protein is deposited in all the wells of the plate and incubated for 2 h at room temperature, stirring gently. Then the primary antibodies directed either against podocalyxin (Santacruz, Sc-23904) at 1/500, or against the CD41 platelet marker (Novus, MAB 7616) at 1/500, are brought into contact with the microvesicles captured in the wells for 2 h at room temperature with gentle horizontal agitation. After two washing operations, the secondary antibodies coupled to the enzyme HRP, at 1/5000 dilution, are incubated for 1 h at room temperature with gentle horizontal agitation. After six washing operations, 100 μL of chromogenic developer TMB (3,3′,5,5′-tetramethylbenzidine) is deposited in the wells for a duration of 30 minutes at room temperature with gentle horizontal agitation. The reaction is stopped by adding 100 μL of sulfuric acid, and the absorbance of each well is measured using a spectrophotometer at 450 nm.

Results

a/ Identification of Protein Markers Carried by Microvesicles

Validation of the Biomarker Podocalyxin in the Microvesicles Obtained from the Urine of Diabetic Patients According to the Stage of Nephropathic Complication by the Western Blot Technique

Podocalyxin is a transmembrane protein expressed by podocytes, glomerular cells that are only present in the kidney; it is described in the literature as a marker of cellular lesion of the podocytes.

The protein beta-actin is a protein of the cytoskeleton expressed in all cells; this stable, universal expression means that it is often used in Western blot as a reference protein when it is necessary to quantify the level of a protein whose expression may vary as a function of the disease.

The protein annexin-A5 is localized near the plasma membrane in all cells; like beta-actin, it is also used as a reference protein in Western blot. Accordingly, analysis of the expression of the protein podocalyxin by Western blot in the microvesicles obtained from the urine of healthy donors or of diabetic patients will therefore rather be expressed in the form of a ratio: podocalyxin/beta-actin ratio and podocalyxin/annexin-A5 ratio.

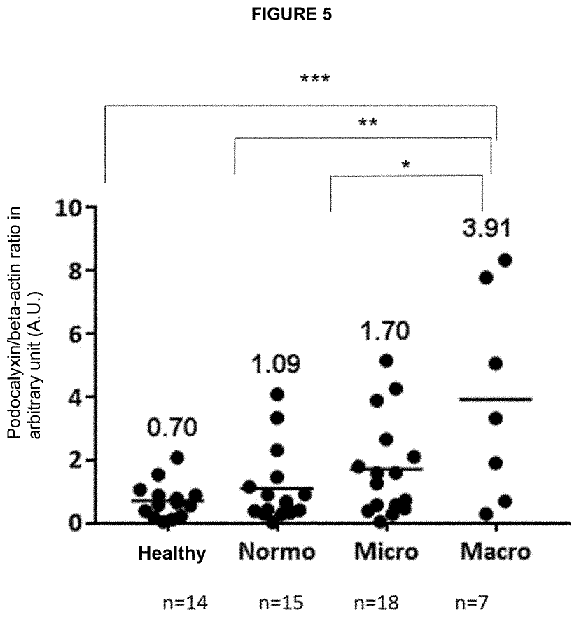

FIG. 5 shows the podocalyxin/beta-actin ratio. Each point represents the ratio obtained after Western blot analysis of the urine microvesicle sample obtained from a healthy donor or from a diabetic patient. It can be seen that the healthy donors express a low podocalyxin/beta-actin ratio, on average 0.70, this corresponds to the baseline level of natural production of microvesicles derived from podocytes in nonpathological conditions. This ratio gradually increases according to the stages of nephropathy linked to diabetes. Thus, it can be seen that the podocalyxin/beta-actin ratio reaches on average 1.09 in the normoalbuminuric patients, then 1.70 in the microalbuminuric patients and finally 3.91 in the macroalbuminuric patients. ANOVA statistical analysis, with the Tukey inter-group comparison test, using the GraphPad Prism software, reveals a significant difference between the groups: Healthy versus Macro with a p-value: p<0.001, Normo versus Macro with p<0.01 and Micro versus Macro with p<0.05.

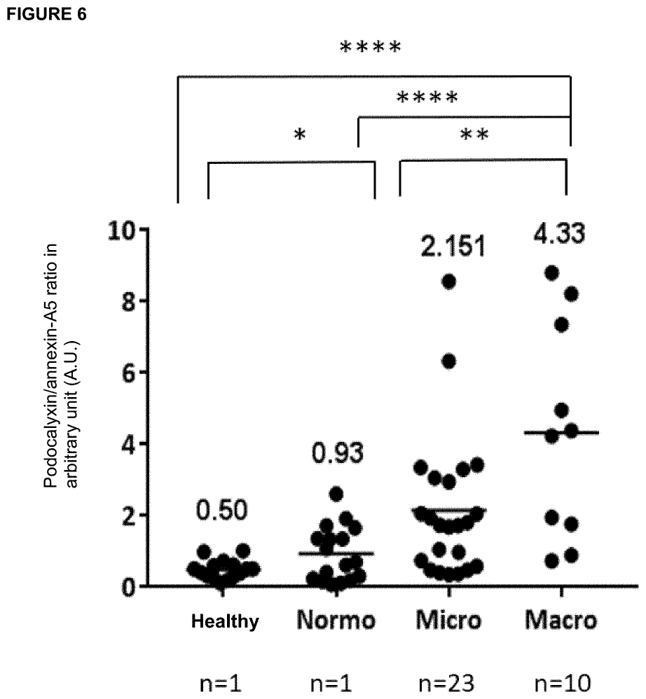

FIG. 6 shows the podocalyxin/annexin-A5 ratio. Each point represents the ratio obtained after Western blot analysis of the urine microvesicle sample obtained from a healthy donor or from a diabetic patient. As before, it can be seen for the podocalyxin/beta-actin ratio that the healthy donors express a low podocalyxin/annexin-A5 ratio, on average 0.50; this corresponds to the baseline level of natural production of microvesicles derived from podocytes in nonpathological conditions. Once again, the ratio gradually increases according to the stages of nephropathy linked to diabetes. Thus, it can be seen that the podocalyxin/annexin-A5 ratio changes on average to 0.93 in the normoalbuminuric patients, then to 2.15 in the microalbuminuric patients and finally to 4.33 in the macroalbuminuric patients. ANOVA statistical analysis, with the Tukey inter-group comparison test, using the GraphPad Prism software, reveals a significant difference between the groups: Healthy versus Normo with p<0.05, Healthy versus Macro with p<0.0001, Normo versus Macro with p<0.0001 and Micro versus Macro with p<0.01.

In conclusion, it may be envisaged that the podocalyxin/annexin-A5 ratio could be used in the context of a kit for detecting nephropathic complications in the microvesicles derived from patients' urine. When this ratio increases, this is evidence of a cellular lesion of the podocytes.

Detection of the Platelet Marker, CD41, in the Microvesicles Derived from the Plasma of Diabetic Patients According to the Stage of Nephropathic Complication by the Western Blot Technique

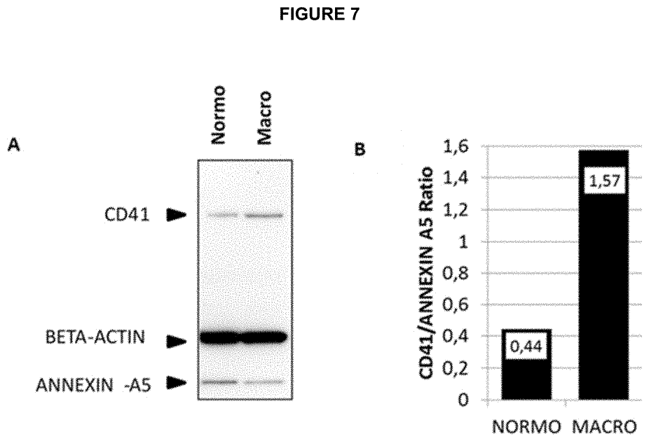

The purpose of our experiments in this part is to show that we are able to detect a marker in the microvesicles purified by centrifugation obtained from human plasma. For this, we developed detection of CD41, a protein localized on the surface of the membranes of the platelets, by the Western blot technique. The microvesicles that originate from the budding of the membranes of the platelets express CD41. Thus, we analyzed the CD41/annexin-A5 expression ratio in the microvesicles derived from the plasma of a Normoalbuminuric diabetic patient and of a Macroalbuminuric diabetic patient (FIG. 7 below). We are able to observe specific detection of the CD41 platelet marker in the protein extracts from microvesicles derived from the plasma by the Western blot technique. The CD41/annexin-A5 ratio varies from one individual to another. This experiment shows that it is possible for a marker carried by the circulating microvesicles to be quantified in the plasma.

Conclusion

We have shown that we are able to quantify protein biomarkers in the microvesicles isolated from different biological fluids: urine and plasma.

Moreover, the podocalyxin/annexin-A5 ratio could be used in a diagnostic kit for detecting a nephropathic complication.

b/ Validation of the Surface of DNA-BIND® Polystyrene Grafted with Complex C1

Capture of Model Microvesicles and Enzymatic Detection of Biomarkers Carried by these Microvesicles

Microvesicles carry biological material such as enzymes, proteins with biological activity. We employed the enzymatic technique described in patent PCT/FR2012/050610 to validate grafting of complex C1 on the surface of DNA-BIND® polystyrene. The model microvesicles are captured by the material with an amount of 2.5 μg of total microvesicles (protein equivalent) in 25 μL for 1 h at room temperature. The enzymatic activity present in each experimental condition is analyzed by enzyme kinetics. That is, the rate of appearance of a chromogenic product, CBS0065, catalyzed by the enzyme will be monitored for 18 h. The experimental conditions are as follows:

Unrinsed condition 100%: this is the control condition, the model microvesicles are left in the wells for 1 h and the enzymatic activity is determined directly without rinsing the wells. The enzymatic activity detected is the maximum activity of the extract of model microvesicles. This condition therefore represents the positive control, 100%. Rinsed condition without complex: the model microvesicles are incubated for 1 h in the wells of the plate that has not been grafted with complex C1, then rinsed three times and the enzymatic activity is analyzed. This condition represents the negative control; the enzymatic activity obtained is evidence of nonspecific interaction of the model microvesicles.

Rinsed condition with complex: the model microvesicles are incubated for 1 h in the wells of the plate grafted with complex C1, then rinsed three times and the enzymatic activity is analyzed. This condition represents capture of the microvesicles by the complex; the enzymatic activity obtained is evidence of specific interaction of the model microvesicles.

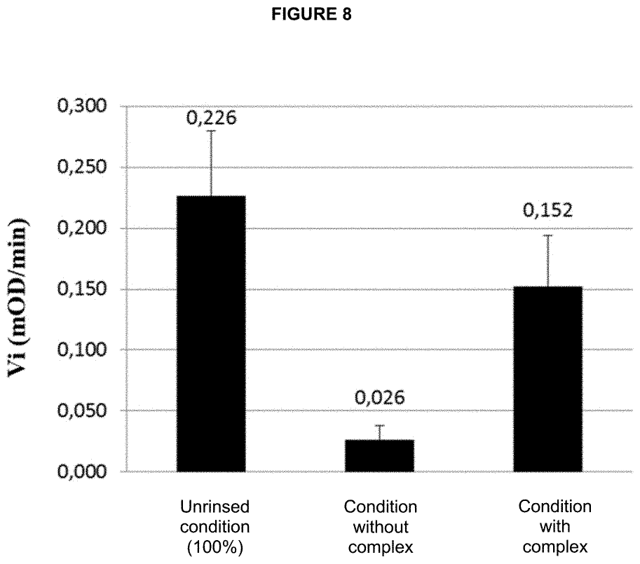

FIG. 8 shows the mean values of Vi (mean value of the calculated initial rate) obtained for each condition described above. A mean value of Vi of 0.226 mOD/min is observed in the control condition of the unrinsed microvesicles, which represents the maximum mean value of Vi obtained for 2.5 μg of microvesicles, this is therefore 100%. A mean value of the initial rate of 0.026 mOD/min is measured in the wells where the model microvesicles are incubated on a support not grafted with the metal complex. The degree of nonspecific interaction is: 0.026/0.226=0.115, or 11.5%. A mean value of the initial rate of 0.152 mOD/min is measured in the wells where the microvesicles are incubated on a support grafted with the metal complex C1 and then rinsed. The specific capture rate is therefore: 0.152/0.226=0.672, or 67.2%. In conclusion, this experiment shows (1) that the surface of DNA-BIND® polystyrene grafted with complex C1 allows capture of the microvesicles of HUVEC cell models and (2) that the surface of DNA-BIND® polystyrene grafted with complex C1 allows quantification of the enzymes carried by the model microvesicles. Moreover, specific capture of 67.2% of the microvesicles can be evaluated.

Capture of the Microvesicles Isolated from Human Urine and ELISA Detection of a Podocyte Marker Carried by these Microvesicles

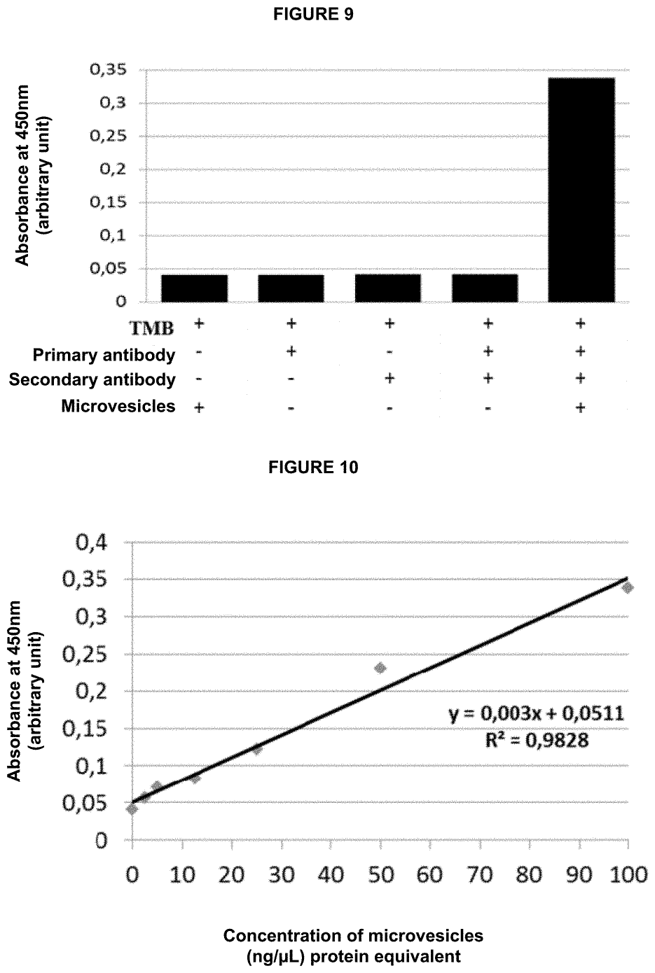

We tested for the presence of podocalyxin and detection thereof by the ELISA immunologic method to validate specific capture of the microvesicles isolated from urine and to demonstrate the capacity for quantification of a biomarker after capture on our material. To do this, we incubated the microvesicles isolated by centrifugation from urine overnight at 4° C. with gentle agitation on the DNA-BIND® Costar 96-well polystyrene plate grafted entirely with complex C1. We deposited a maximum quantity of microvesicles equivalent to 20 μg of proteins assayed by spectrophotometry (i.e. 100 ng/μL in the 200 μL of final reaction). After several washing operations, we detected the presence of the protein podocalyxin using an antibody specifically directed against it. A secondary antibody coupled to the enzyme horseradish peroxidase is added in order to amplify and detect a signal by spectrophotometry at 450 nm in the presence of a substrate with chromogenic properties, TMB. The results are reported in FIG. 9. A specific signal can thus be observed in the condition: Microvesicles captured by the material+primary antibody against podocalyxin+secondary antibody. A signal equivalent to the background noise is observed in all the other conditions corresponding to the various controls. It can be concluded that the microvesicles isolated from human urine were captured specifically and that podocalyxin on the surface of the microvesicles is detected specifically.

In order to demonstrate that ELISA detection of the biomarker of the protein podocalyxin in the microvesicles derived from human urine after capture on materials can be regarded as semiquantitative, we carried out a range of deposits of the microvesicles comprised between 0.5 μg and 20 μg, i.e. at concentrations comprised between 2.5 ng/μL and 100 ng/μL in 200 μL of final reaction. The results of the measurements of absorbances as a function of the different quantities of microvesicles are presented in FIG. 10. A linear relation can be observed between the concentration of microvesicles deposited and captured by the material and the signal detected by the ELISA technique for the protein podocalyxin.

Thus, the material (complex C1 grafted on the DNA-BIND® polystyrene plate from Costar) not only allows specific capture of the microvesicles isolated from urine, but also makes it possible to quantify the presence of podocalyxin, by the ELISA technique with a threshold of detection of 2.5 ng/μL of microvesicles deposited.

Capture of the Microvesicles Isolated from Human Plasma and ELISA Detection of the CD41 Platelet Marker Carried by these Circulating Microvesicles

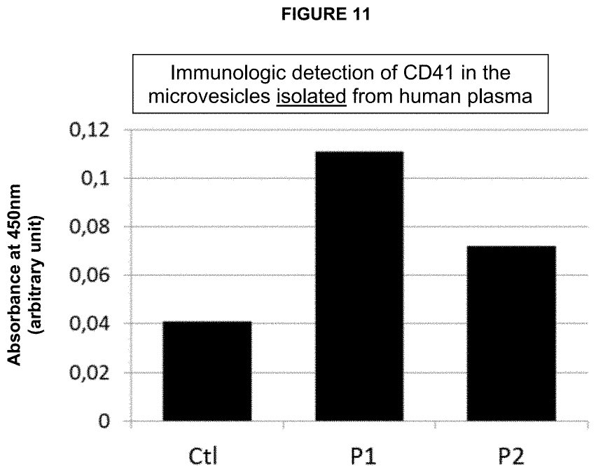

We wanted to show that the material (complex C1 grafted on the DNA-BIND® polystyrene plate from Costar) is also capable of capturing the circulating microvesicles (i.e. isolated from human plasma) isolated beforehand by centrifugation and that it is possible to quantify, by the ELISA technique, the presence of a protein on the captured microvesicles. For this, we selected the protein CD41 that is present on the surface of the platelets and is therefore found on the surface of the microvesicles derived from their membrane budding. The circulating microvesicles isolated beforehand by centrifugation from plasma from normoalbuminuric diabetic patients were deposited in the polystyrene plate functionalized with complex C1 and incubated overnight at 4° C., with gentle agitation. A quantity of microvesicles isolated from 1.5 mL of plasma was deposited per well. After several washing operations, we detected the presence of the protein CD41 by means of an antibody specifically directed against it. A secondary antibody coupled to the enzyme horseradish peroxidase is added in order to amplify and detect a signal by spectrophotometry at 450 nm in the presence of a substrate with chromogenic properties, TMB. The results are reported in FIG. 11. A specific signal is observed in the wells in which the microvesicles of plasma from patients (P1 and P2) were deposited. These results indicate not only specific capture of the circulating microvesicles but also the possibility of quantifying, by the ELISA technique, the presence of a protein on their surface, in this case the platelet protein CD41.

Capture of the Microvesicles Contained in Human Plasma and Detection of the Platelet Protein Biomarker, CD41, by ELISA

Finally, we wanted to show that the material (complex C1 grafted on the DNA-BIND® polystyrene plate from Costar) is capable of capturing the unisolated microvesicles. Therefore in this case the sample is not microvesicles isolated beforehand by centrifugation, but is obtained directly from human plasma containing microvesicles. We tried to quantify the presence of the protein CD41 carried by the microvesicles of the plasma sample, using the ELISA technique. For this, we incubated 300 μL of plasma from normoalbuminuric diabetic patients overnight at 4° C. with gentle agitation, on the DNA-BIND® polystyrene plate grafted with complex C1. After several washing operations, we detected the presence of the protein CD41 by means of an antibody specifically directed against it. A secondary antibody coupled to the enzyme horseradish peroxidase is added in order to amplify and detect a signal by spectrophotometry at 450 nm in the presence of a substrate with chromogenic properties, TMB. The results are reported in FIG. 12. Although the levels are low, we observe a signal for detection of CD41 in the wells in which the plasmas from patients (P3, P4 and P5) were deposited in the wells grafted with complex C1. These results indicate not only specific capture of the microvesicles (not purified beforehand) present in the plasma but also the possibility of quantifying, by the ELISA technique, the presence of a protein on their surface, in this case the platelet protein CD41.

### Example 5: Early Diagnosis of a Diabetic Nephropathy by Capture of Cellular Microvesicles on a Solid Support

Material and Methods
Samples of human biological fluids: All the samples were stored at −80° C. until used.

Human urine samples from healthy subjects: The human urine samples were collected at the Institute of Chemistry and Biology of Membranes and Nano-objects (CBMN) at Pessac from healthy subjects during the day in conventional urine pots without adding additives. The start of urination was not collected, and the samples were stored at −80° C. until used.

Human urine samples from diabetic patients: The donors are diabetic patients with nephropathy at the normoalbuminuric stage (Normo), microalbuminuric stage (Micro) or macroalbuminuric stage (Macro). These stages correspond to the severity of the disease, the Macro stage being the most advanced. The urine samples were collected during the day in conventional urine pots without adding additives. Moreover, the start of urination was not collected.

Purification of the Microvesicles

Microvesicles derived from a cell model: The human umbilical vein endothelial cells, HUVECs (Promocell; ref. C-12208), were cultured in complete medium (Promocell; Endothelial Cell Growth Medium 2 Kit; ref. C-22111) in accordance with the supplier's recommendations. Between passages 3 and 6, the cells were rinsed twice in PBS 1×, then activated with a solution of TNF-alpha (Peprotech; ref. 300-01A) prepared at 100 ng/mL in complete medium for 24 h at 37° C. in order to produce microvesicles. Then the supernatant containing the microvesicles was transferred to a 50 mL tube. First, the cellular debris and apoptotic bodies were sedimented by centrifugation at 12 000 g for 2 min at 4° C. (SIGMA; thermostatically controlled centrifuge 3-18KHS) in order to remove them. Secondly, the supernatant containing the microvesicles was transferred to a new 50 mL tube and the microvesicles were sedimented by centrifugation at 20 000 g for 1.5 h at 4° C. The supernatant was removed and the pellet was rinsed with 1 mL of buffer solution (HEPES 10 mM; NaCl 0.15 M; pH 7.4). The solution of microvesicles was transferred to a new 1.5 mL microtube and centrifuged at 20 000 g for 1.5 h at 4° C. The supernatant was removed and the pellet of microvesicles was rinsed again and then centrifuged at 20 000 g for 1.5 h at 4° C. This last pellet of microvesicles was finally resuspended in buffer solution (HEPES 10 mM; NaCl 0.15 M; pH 7.4) and this solution was stored at −80° C. until used.

Microvesicles derived from human urine samples: In order to isolate the microvesicles, the urine was thawed overnight at 4° C. and 40 mL was centrifuged at 1500 g for 15 min at 4° C. The supernatant was transferred to a new tube and centrifuged at 12 000 g for 2 min at 4° C. to remove the cellular debris and apoptotic bodies. The supernatant was then transferred to a new tube and centrifuged at 20 000 g for 1.5 h at 4° C. in order to sediment the microvesicles. The supernatant was removed and the pellet was rinsed in 1 mL of buffer (HEPES 10 mM; NaCl 0.15 M; pH 7.4). The solution of microvesicles was transferred to a 1.5 mL microtube and then centrifuged at 20 000 g for 1.5 h at 4° C. The supernatant was removed and the pellet of microvesicles was rinsed again and then centrifuged at 20 000 g for 1.5 h at 4° C. This last pellet of microvesicles was finally resuspended in buffer solution (HEPES 10 mM; NaCl 0.15 M; pH 7.4) and this solution was stored at −80° C. until used.

CRYOMEB Observation

Capture of the microvesicles: a buffer solution (HEPES 10 mM; NaCl 0.15 M; pH 7.4) containing microvesicles (derived from cellular activation of the HUVEC cells or derived from a human urine sample from a healthy subject) was incubated overnight at room temperature, at a rate of 300 μL per material, and then removed by 10 successive rinsings with the buffer solution. The materials used are the 1 cm² square of PVC+C1 and the “DNA-BIND®” plate well+C1.

CRYOMEB observation: The samples were observed with a “Quanta 250 FEG FEI” field-effect scanning electron microscope (SEM) equipped with a “Quorum PP3000T” cryo module. The samples were first glued to a support using a Tissue-Tek®/carbon mixture, and then cooled rapidly in pasty nitrogen. They were then put in the first chamber of the microscope to undergo sublimation for 20 min at −95° C. and then in the second chamber to undergo metallization by spraying platinum under argon at 10 mA for 1 min. Finally, the metallized samples were placed in the observation chamber for image acquisition.

Capture of the Microvesicles on DNA-BIND® Support

Assay of the proteins: The proteins were assayed by NanoDrop (ND1000; Thermo Fisher SCIENTIFIC) in order to determine the quantity of microvesicles present in each solution.

Capture of the microvesicles: the equipment used, also called “kit” in the present application, is the DNA-BIND® 96-well plate (COSTAR; ref. 2525) functionalized with complex C1. The experiment is divided into two parts: a part with capture of the microvesicles by the kit and a part with control microvesicles away from the plate and thus not captured by the kit. The “control” part informs us about the initial composition of the microvesicles without capture. A buffer solution (HEPES 10 mM; NaCl 0.15 M; pH 7.4) containing microvesicles is then separated into two equal parts: a part for capture and a part for the controls.

Part for capture of the microvesicles: The buffer solution (HEPES 10 mM; NaCl 0.15 M; pH 7.4) containing microvesicles was incubated on the materials for 1 h at 37° C. or overnight at 4° C. at a rate of 50 μL per well. The plate was covered with a film to prevent any evaporation. The buffer solution was then removed and the wells were rinsed three times for 10 min with the same buffer formula at a rate of 200 μL/well in order to isolate the captured microvesicles from the other objects present in solution and remove those that would be adsorbed nonspecifically on the material. The microvesicles were then lysed in 30 μL of lysis buffer (99% in RIPA buffer—1% of cocktail of protease inhibitors) per well for 30 min in ice in order to recover all the proteins from the microvesicles individually. After homogenization, the solution was transferred to a 1.5 mL microtube. The membrane debris was sedimented by centrifugation at 10 000 g for 10 min at 4° C. in order to remove it. The supernatant containing the proteins was recovered in a new microtube.

Part for control microvesicles: The buffer solution (HEPES 10 mM; NaCl 0.15 M; pH 7.4) containing microvesicles was incubated in a 1.5 mL microtube for 1 h at 37° C. or overnight at 4° C. at a rate of 50 μL per microtube. After homogenization of the solution, the microvesicles were sedimented by centrifugation at 20 000 g for 1.5 h at 4° C. The supernatant was removed by aspiration. The microvesicles were then resuspended and lysed in 30 μL of lysis buffer per microtube for 30 min in ice in order to recover all the proteins from the microvesicles individually. After homogenization of the solution, the membrane debris was sedimented by centrifugation at 10 000 g for 10 min at 4° C. in order to remove it. The supernatant containing the proteins was recovered in a new microtube.

Analysis by Western Blot:

Each sample (protein extract) was prepared in a microtube with loading buffer (Bolt™ LDS Sample Buffer 4X+Bolt™ Sample Reducing Agent 10X) at a rate of 19.5 μL of solution of proteins to 30 μL of final sample in the proportions indicated by the supplier. The samples were vortexed and quickly centrifuged with a benchtop centrifuge before being denatured at 95° C. for 5 min. The whole sample (30 μL) was deposited on acrylamide gel in denaturing conditions. A size marker was always added as reference. The samples migrated for 10 min at 50 V and then for 1.5 h at 120V. The proteins present in the gel were transferred onto PVDF membrane by semiliquid transfer for 15 min at constant 1.3 A. The nonspecific sites of the membrane were saturated in a bath of milk buffer (5% of skim milk in buffer Tris Buffer Saline (TBS)—Tween 0.1%) for 1 h at room temperature with stirring. After two rinsings of 10 min in TBS—Tween 0.1% buffer with stirring, the membrane was incubated in a solution of primary antibodies, prepared in milk buffer, overnight at 4° C. with stirring. The antibodies (Ab) used are Ab anti-podocalyxin (Santacruz, ref. Sc-23904; dilution 1/2000), Ab anti-beta-actin (Sigma, ref. A1978; dilution 1/5000) and Ab anti-annexin-A5 (Sigma, ref. A8604; dilution 1/2000). After two rinsings of 10 min in TBS—Tween 0.1% buffer with stirring, the membrane was incubated in a solution of secondary Ab, prepared in milk buffer, for 1 h at RT with stirring. The signal was detected by a gel imager (GeneGnome, SYNGENE) by means of a chemiluminescence reaction after incubation of the membrane with a developer in Pico for 5 min (ThermoFisher SCIENTIFIC, ref. 34580) or in Femto for 2 min (ThermoFisher SCIENTIFIC, ref. 34094) depending on the signal intensity. Image acquisition was carried out with the GeneSys software. An analysis, both qualitative and quantitative, was carried out using Image J. The qualitative aspect was assessed by the presence of bands, at the molecular weight specific to each marker of interest, as well as from the intensity of their signal. The quantitative aspect was determined by calculating a ratio: intensity of the signal of the pathological marker, podocalyxin, relative to the intensity of the signal of the reference marker of the microvesicles, annexin-A5. This ratio makes it possible to determine the quantity of pathological marker relative to the number of microvesicles.

Results

CRYOMEB Analysis

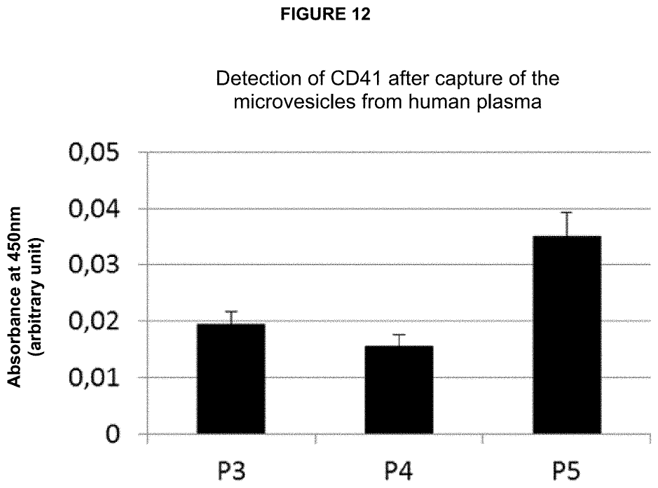

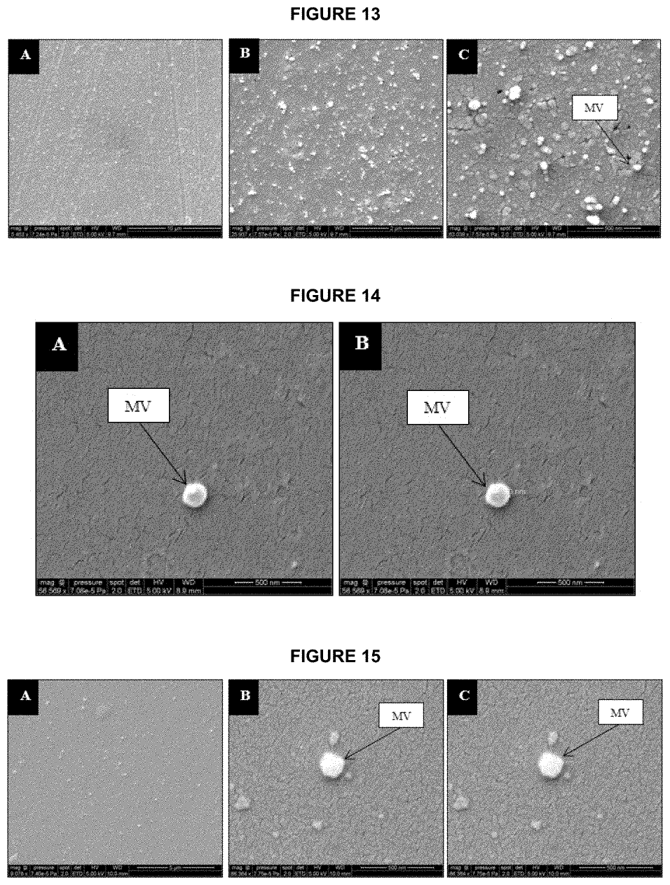

The images of capture of the microvesicles by PVC+C1 are shown in FIG. 13 (microvesicles derived from cellular activation of the HUVEC cells) and the images of capture by DNA-BIND®″+C1 are shown in FIG. 14 (microvesicles derived from cellular activation of the HUVEC cells) and FIG. 15 (microvesicles derived from a human urine sample from a healthy subject). These images show the presence of numerous spherical objects from 40 nm to 100 nm in diameter for PVC+C1 (FIG. 13) and of 150 nm on average on DNA-BIND®″+C1 (FIG. 14 and FIG. 15) and therefore of phosphatidylserine-positive microvesicles captured by the different materials tested. The 96-well plates functionalized with C1 capture the microvesicles derived from a cell model (HUVEC).

Capture of the Microvesicles and Detection by Western Blot

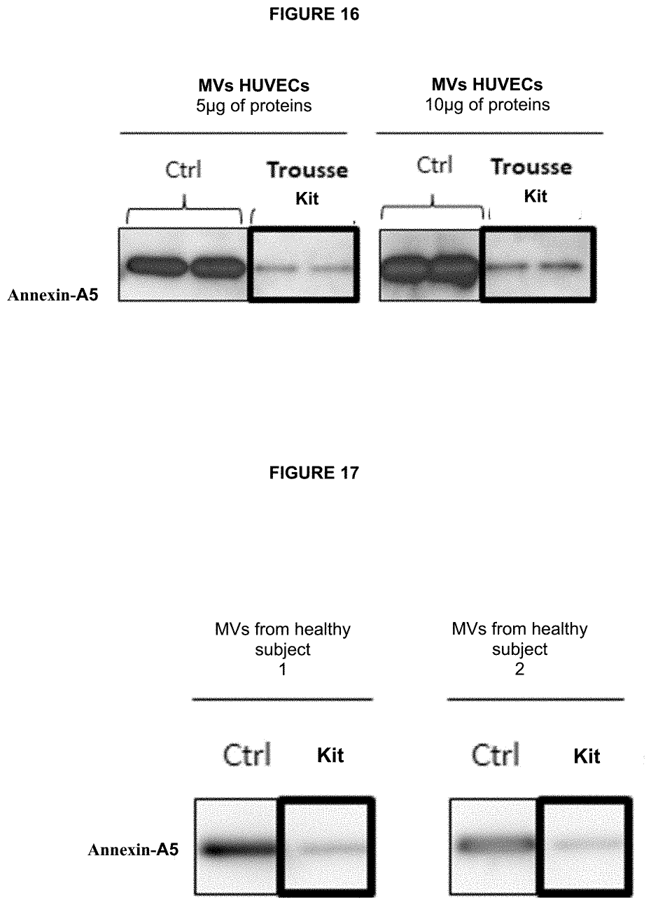

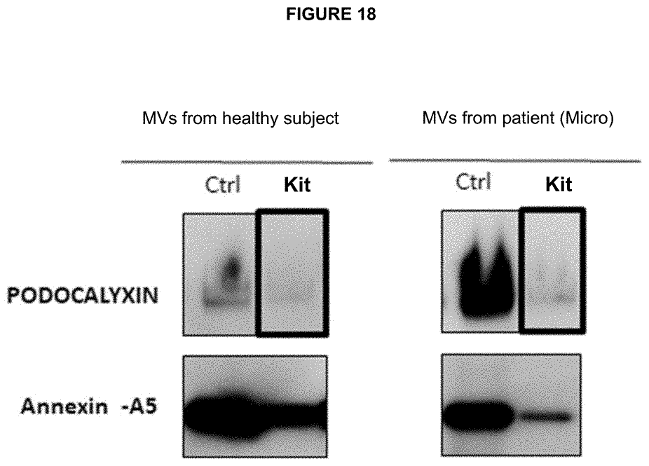

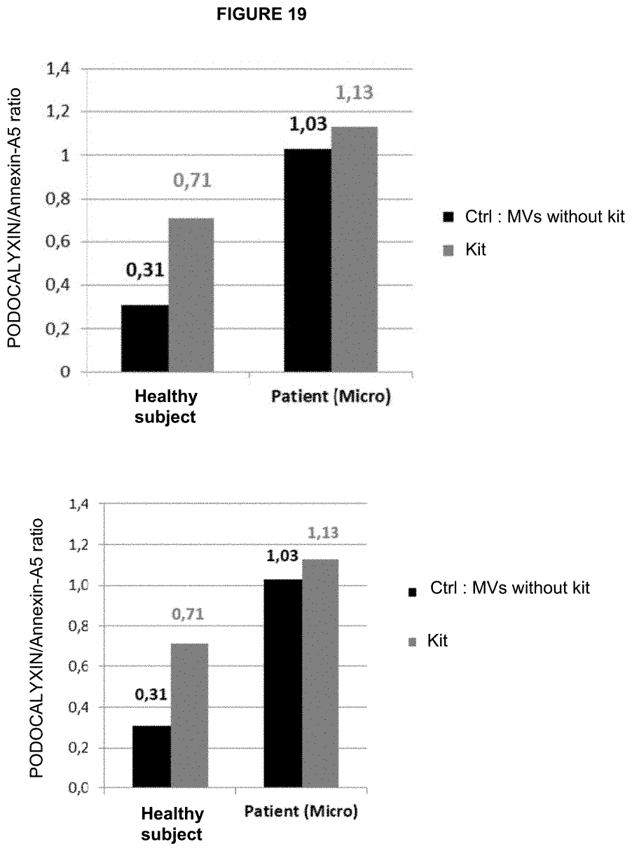

First, capture of the microvesicles by the kit was demonstrated by detection of the reference marker annexin-A5 carried by model microvesicles, after incubation on the kit at two different doses. The results are shown in FIG. 16. The presence of a significant band at the molecular weight corresponding to annexin-A5 after capture provides evidence of the specific and effective recruitment of the microvesicles. Moreover, the change in intensity of the signal as a function of the quantity of microvesicles deposited indicates a dose-response that is also specific. Secondly, capture of the microvesicles by the kit was demonstrated by detection of annexin-A5 on microvesicles derived from human urine samples from healthy subjects. The results are shown in FIG. 17. The presence of a significant band at the molecular weight of annexin-A5 after incubation on the kit is evidence of specific capture of the urinary microvesicles. After validation of capture, we tested detection of a pathological biomarker on the microvesicles captured by the kit. The biomarker studied is podocalyxin, providing evidence of nephropathy at the podocyte level. The microvesicles used are derived from urine samples from a healthy subject and from a diabetic patient with nephropathy at the microalbuminuric stage (Micro). The results are shown in FIG. 18 and FIG. 19. The data relating to the donors are indicated in Table 5.

TABLE 5
Clinical data relating to the donors shown in FIG. 18 and FIG. 19.

| Type of donor | Diabetic patient | Healthy subject |
|---|---|---|
| Donor identification number | Micro 1 | Healthy 1 |
| Albuminuria stage | Micro | / |
| Microalbuminuria assay (mg/mmol) | 8.8 | / |
| Microalbuminuria assay (mg/day) | 58.6 | / |
| Date of birth | May 1, 1981 | / |
| Age (years) | 36 | 25 |
| Sex | Male | Male |
| Type of diabetes | DT2 | / |
| Year discovered | 2009 | / |
| Duration of diabetes (years) | 8 | / |
| Weight (kg) | 115.1 | 68 |
| Height (cm) | 171 | 178 |
| BMI (body mass index, kg/m²) | 39.3 | 21 |
| BP (blood pressure) (mmHg) | 140/95 | / |
| Macroangiopathy | Yes | / |
| HbA_{1c} (glycosylated hemoglobin, %) | 7 | / |
| FBG (fasting blood glucose) | 0.88 | / |
| Creatinine (μmol/L) | 134 | / |
| GFR (glomerular filtration rate, | 67 | / |
| mL/min) | | |
| Hematuria | No* | / |
| Leukocyturia | No* | / |
| Statin | Yes* | / |
| RAAS (renin angiotensin aldosterone | Yes* | / |
| system) blocker | | |
| Diuretic | No* | / |
| Aldactone | No* | / |
| Grading of foot ulceration risk | 0 | / |

*(Yes: the patient is receiving the treatment; No: the patient is not receiving the treatment)

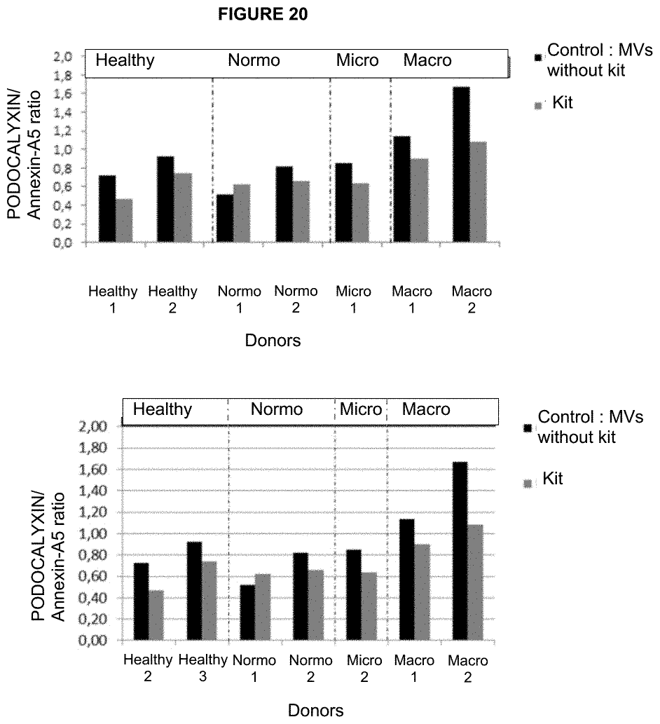

The presence of the characteristic bands of podocalyxin and of annexin-A5 after capture on the kit, demonstrated in FIG. 18, shows that Western blot makes it possible to detect a pathological marker after capture on the kit. Moreover, quantification of the signal, illustrated in FIG. 19, shows that the podocalyxin/annexin-A5 ratio, which appears to reveal the disease, is of the same order of magnitude on the microvesicles without the kit (shown in black) and on those that are captured by the kit (shown in gray). This result shows that the population of microvesicles captured is representative of the population initially present in the biological fluid, allowing a relevant diagnosis. Finally, we compared the podocalyxin/annexin-A5 ratio of the microvesicles derived from urine samples from healthy subjects and from diabetic patients with nephropathy at the various stages of the disease (Normo, Micro, Macro) without capture and after capture by the kit. The results are shown in FIG. 20 and the clinical information relating to the donors is given in Table 6.

TABLE 6
Clinical data relating to the donors shown in FIG. 20

| | Healthy | Healthy | Diabetic | Diabetic | Diabetic | Diabetic | Diabetic |
|---|---|---|---|---|---|---|---|
| Type of donor | subject | subject | patient | patient | patient | patient | patient |
| Donor | Healthy | Healthy | Normo 1 | Normo 2 | Micro 2 | Macro 1 | Macro 2 |
| identification | 2 | 3 | | | | | |
| number | | | | | | | |
| Albuminuria stage | / | / | Normo | Normo | Micro | Macro | Macro |
| Microalbuminuria | / | / | 0 | 0 | 8.7 | 260.4 | 368 |
| assay (mg/mmol) | | | | | | | |
| Microalbuminuria | / | / | 0 | 0 | 77.2 | 2864 | 3275 |
| assay (mg/day) | | | | | | | |
| Date of birth | / | / | 18 May 1949 | 3 Dec. 1975 | 28 Mar. 1982 | 22 Jan. 1982 | 17 Mar. 1946 |
| Age (years) | 27 | 25 | 67 | 41 | 75 | 35 | 71 |
| Sex | Female | Female | Female | Male | Male | Male | Female |
| Type of diabetes | / | / | DT1 | DT1 | DT2 | DT1 | DT1 |
| Year discovered | / | / | 2014 | 2006 | 2007 | 1993 | 2007 |
| Duration of | / | / | 3 | 11 | 10 | 26 | 11 |
| diabetes (years) | | | | | | | |
| Weight (kg) | 55 | 53 | 42 | 89 | 121 | 62.2 | 96 |
| Height (cm) | 160 | 161 | 164 | 175 | 168 | 180 | / |
| BMI (body mass | 21 | 20 | 15.6 | 29 | 42.8 | 19 | 34.1 |
| index, kg/m²) | | | | | | | |
| BP (blood | / | / | 137/82 | 110/60 | 120/60 | 137/84 | 155/83 |
| pressure) (mmHg) | | | | | | | |
| Macroangiopathy | / | / | No | No | No | No | No |
| HbA_{1c} | / | / | 11.5 | 9 | 8 | 7.3 | 8.1 |
| (glycosylated | | | | | | | |
| hemoglobin, %) | | | | | | | |
| FBG (fasting blood | / | / | 2.34 | 1.4 | 1.51 | 1.66 | 1.62 |
| glucose) | | | | | | | |
| Creatinine | / | / | 70 | 72 | 75 | 207 | 44 |
| (μmol/L) | | | | | | | |
| GFR (glomerular | / | / | 77 | 110 | 85 | 35 | 98 |
| filtration | | | | | | | |
| rate, mL/min) | | | | | | | |
| Hematuria | / | / | N | / | Yes | / | / |
| Leukocyturia | / | / | N | / | No | / | / |
| Statin | / | / | O | N | Yes | Yes | Yes |
| RAAS (renin | / | / | N | N | Yes | Yes | Yes |
| angiotensin | | | | | | | |
| aldosterone | | | | | | | |
| system) blocker | | | | | | | |
| Diuretic | / | / | N | N | Yes | Yes | Yes |
| Aldactone | / | / | N | N | Yes | No | Yes |
| Grading of foot | / | / | 0 | / | 1 | / | / |
| ulceration risk | | | | | | | |

* (Yes: the patient is receiving the treatment; No: the patient is not receiving the treatment)

The diagram in FIG. 20 shows that the ratios calculated from the microvesicles after capture on the kit (in gray) are representative of the ratios calculated from the microvesicles without capture (in black) regardless of the stage of the disease (Normo, Micro or Macro). The Western blot detection technique therefore makes it possible to validate that the kit can be used for making an early diagnosis and for assessing the severity of a disease.

1-12. (canceled)

13. A solid support comprising, grafted on its surface, a compound of the following formula (I) or (II): 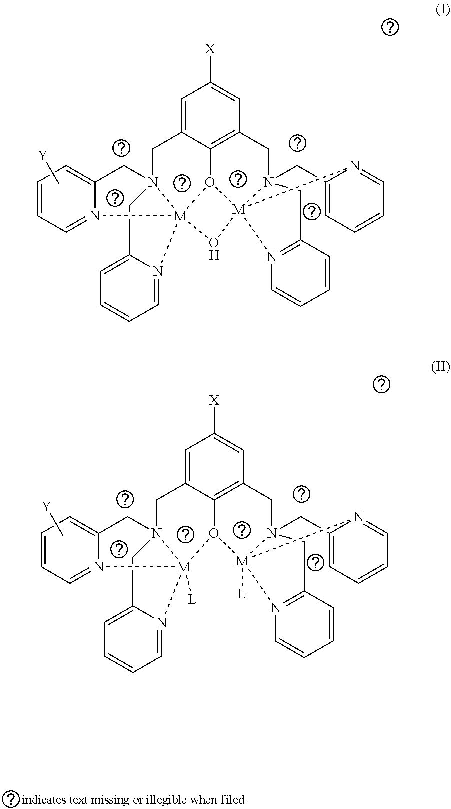
in which
M⁺ⁱ represents a metal ion and i is 1, 2 or 3;
L represents an exchangeable ligand;
X represents a group —(CH₂)ₘNH₂, or a group —CH₂—NHC(O)—R—NH₂ in which R is a C₂-C₁₀ alkyl group, substituted or unsubstituted, linear or branched;
m=1 to 12;
n=1, 2 or 3; and
Y represents H or (CH₂)ₚ—NH₂, where p=0 to 12.

14. The solid support according to claim 13, said solid support being characterized in that it is a solid support made of poly(vinyl chloride) (PVC), poly(ethylene terephthalate) (PET) or polystyrene (PS).

15. The solid support according to claim 13, characterized in that it is a solid support of PET prefunctionalized by means of NHS functions, of PVC functionalized by means of glutaraldehyde, or of PS functionalized by means of N-oxysuccinimide functions.

16. The solid support according to claim 13, characterized in that M is selected from the group consisting of Zn, Cu, Mn, Co, Ni and Fe.

17. The solid support according to claim 13, characterized in that:
X represents —(CH₂)m-NH₂, m=1, n=1 and Y represents H; or
X represents —(CH₂)m-NH₂, m=1, n=2 and Y represents H; or
X represents —CH₂—NHC(O)—R—NH₂, R represents C₅H₁₀, n=1 or 2 and Y represents H.

18. The solid support according to claim 13, characterized in that the grafted compound is a compound of formula (I), selected from the group consisting of:

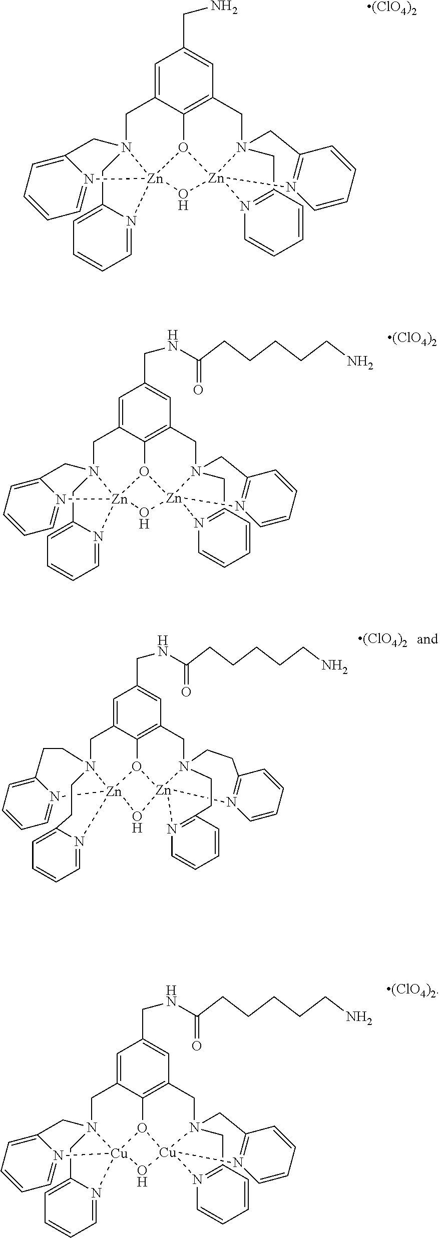

19. A method for capturing microvesicles present in a sample of biological fluid from a subject, comprising bringing said sample into contact with a support according to claim 13.

20. A method for characterization of microvesicles present in a sample of biological fluid from a subject, comprising a step of capture of said microvesicles after bringing said sample into contact with a support according to claim 13, and a step of characterization of the captured microvesicles.

21. A method for the diagnosis of a disease, comprising a step of capturing the microvesicles present in a sample of biological fluid from a subject on a support according to claim 13, and detection of the presence or absence of a marker associated with said disease, or measurement of the level of said marker characteristic of said disease.

22. The method according to claim 21, comprising:
detecting the presence or absence of podocalyxin, or measurement of the level of podocalyxin from the microvesicles captured on the support, for the diagnosis of a nephropathy; or
detecting the presence or absence of alpha-synuclein, or measurement of the level of alpha-synuclein from the microvesicles captured on the support, for the diagnosis of Parkinson's disease; or
detecting the presence or absence of CD41, or measurement of the level of CD41 from the microvesicles captured on the support, for the diagnosis of a diabetic nephropathy.

23. A kit comprising:
a support according to claim 13; and
means for detecting or quantifying a marker associated with a disease or for detecting or quantifying podocalyxin, alpha-synuclein or CD41.

24. The kit according to claim 23, further comprising means for detecting or quantifying a normalization marker or for detecting or quantifying annexin-A5 or beta-actin.
